(19) United States
(12) Patent Application Publication
(10) Pub. No.: US 2008/0318960 A1
(43) Pub. Date: Dec. 25, 2008

(54) PAR2-modulating compounds and their use

(76) Inventors: Ethan Burstein, San Diego, CA (US); Anne Eeg Knapp, Frederiksberg C (DK)

Correspondence Address: DUANE MORRIS LLP - San Diego, 101 WEST BROADWAY, SUITE 900, SAN DIEGO, CA, 92101-8285, US

(21) Appl. No.: 11/435,817

(22) Filed: May 16, 2006

Related U.S. Application Data
(60) Provisional application No. 60/685,305, filed on May 26, 2005.

(51) Int. Cl. A61K 31/502; C07D 237/30; A61P 9/00; A61P 29/00; A61P 11/00; A61P 1/00; A61P 19/00; A61P 25/00; A61P 17/00; A61P 27/00; A61P 35/04

(52) U.S. Cl. 514/248; 544/237; 435/29

(57) ABSTRACT

The present invention relates to compounds, and uses thereof, having the chemical formula:

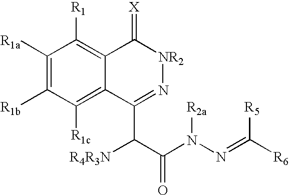

or a pharmaceutically acceptable salt or prodrug thereof, wherein the compound modulates the activity of PAR2 or a PAR2 subtype and thereby may be used to treat or prevent diseases involving abnormal PAR2 or PAR2 subtype activity.

This application relates to and claims the benefit of U.S. Provisional Patent Application Ser. No. 60/685,305.

### FIELD

This invention relates to the fields of organic chemistry, pharmaceutical chemistry, biochemistry, molecular biology and medicine. In particular it relates to compounds that modulate the activity of proteinase-activated receptor-2 (PAR2), to methods of screening for such compounds, to the use of the compounds as tools for the further elucidation of the role of PAR2 in biological systems and to the treatment and prevention of diseases and disorders related to PAR-2 activity.

### BACKGROUND

In 1991, Vu, et al. (Cell, 1991, 64:1057-68) first reported a new receptor in the G-protein-coupled superfamily of receptors that was activated by an unexpected and entirely novel mechanism. The new receptor was found to comprise seven transmembrane domains, three intracellular domains, three extracellular loops, an extracellular N-terminus and a C-tail within the cell. The novel activation mechanism involved the serine protease thrombin, which cleaved the receptor at a specific site in the extracellular N-terminus, thus revealing an N-tethered ligand domain that then intramolecularly bound to and activated the receptor.

It was not until 1994 that it was discovered that Vu's receptor was not one of a kind. In that year, Nystead, et al. (J. Proc. Natl. Acad. Sci (USA), 1994, 91:9208-12) reported a second receptor that was activated by a protease. This receptor was cleaved in its extracellular N-terminus by trypsin to similarly expose an N-tethered ligand that intramolecularly bound and activated the receptor. The discovery prompted the establishment of a new family of receptors, coined the proteinase-activated receptors or PARs.

Since the discovery of the second PAR, two more PARs have been discovered giving four in all. They are designated PAR1, PAR2, PAR3 and PAR4. PAR2 is distinguished from the other three in that it is the only one of the group that is activated by trypsin and tryptase. The other three PARs are all activated by thrombin. Since its discovery, PAR2 has been implicated in a host of physiological and pathophysiological processes. PAR2 is expressed in the cardiovascular system, where it is suggested to play an important role in vascular tone and alterations in vascular function during inflammation with implications in, for example, hypertension. It is expressed in the gastrointestinal system, e.g., the small intestine and colon, as well as in the exocrine organs of the digestive tract, i.e., the stomach, pancreas and salivary glands. It is expressed in the myenteric and submucosal nerve plexuses, the signaling of which may alter the regulation of intestinal motility and secretion. Thus, PAR2 may be implicated in gastrointestinal diseases such as, without limitation, inflammatory bowel disease, irritable bowel disease, pancreatitis and gastritis. PAR2 is expressed in the pulmonary system where its role in the modulation of inflammatory processes suggests it as a pharmacological target for pulmonary diseases such as, without limitation, asthma and chronic obstructive pulmonary disease. PAR2 has also been implicated in the pathology of skin diseases such as, without limitation, cutaneous neurogenic inflammation, pruritis, dry skin syndrome and other inflammatory skin diseases. PAR2 is also strongly expressed in human colon adenocarcinoma cells suggesting a role in cancer. Studies have suggested that PAR2 may play a key role in neurogenic inflammation and pain. It has also been implicated in the genesis of visceral pain.

Given the apparent ubiquity of PAR2 and its participation in diverse physiological and pathophysiological processes in many organ systems including, but not limited to, the cardiovascular system, the pulmonary system, the gastrointestinal tract and the skin and the implication of participation in diseases and disorders therein, it would be extremely valuable to have compounds that are specific agonists and antagonists of PAR2, both as therapeutic agents and as tools to facilitate the continued study of this novel receptor. The present invention provides such compounds.

### SUMMARY

The following are illustrative aspects of the present invention. Additional aspects may be presented in other sections of this application; those in this section are not intended to be, and are not to be construed, as limiting the scope of this invention in any manner whatsoever.

Thus, an aspect of this invention is a compound having the chemical structure:

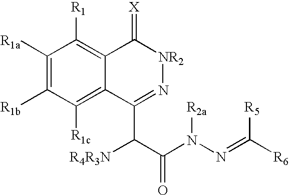

or a pharmaceutically acceptable salt or prodrug thereof, wherein:
R₁, R₁ₐ, R_{1b} and R_{1c} are independently selected from the group consisting of hydrogen, C₁-C₁₀ alkyl, C₃-C₆ cycloalkyl, substituted or unsubstituted aryl, substituted or unsubstituted heteroaryl, hydroxy, nitro, amino, halogen, sulfonate, perhaloalkyl, —OR₇, —NR₇R₇ₐ, —CN, —C(=Z)R₇, —C(=Z)OR₇, —C(=Z)NR₇R₇ₐ, —N(R₇)—C(=Z)R₇ₐ, —N(R₇)—C(=Z)NR₇ₐR_{7b}, —OC(=Z)R₇, and —SR₇, wherein:

Z is oxygen or sulfur;

R₇, R₇ₐ and R_{7b} are independently selected from the group consisting of:
hydrogen;
unsubstituted or substituted C₁-C₁₀ alkyl;
C₁-C₁₀ alkyl substituted with a group selected from the group consisting of aryl and heteroaryl;
unsubstituted C₂-C₁₀ alkenyl;
C₂-C₁₀ alkenyl substituted with a group selected from the group consisting of aryl or heteroaryl;
unsubstituted C₂-C₁₀ alkynyl;
C₂-C₁₀ alkynyl substituted with a group selected from the group consisting of aryl or heteroaryl,
C₃-C₇ cycloalkyl, and
C₅-C₇ cycloalkenyl; or,
   R₁ and R₁ₐ, R₁ₐ and R_{1b} or R_{1b} and R_{1c}, taken together, form a fused aryl or heteroaryl ring;
   X is selected from the group consisting of oxygen, sulfur, nitrogen and NR₈, wherein:
R₈ is selected from the group consisting of C₁-C₄ alkyl, C₃-C₇ cycloalkyl, substituted or unsubstituted aryl, substituted and unsubstituted heteroaryl;
   R₂ and R₂ₐ are independently selected from the group consisting of hydrogen, unsubstituted or substituted C₁-C₄ alkyl, unsubstituted or substituted C₃-C₇ cycloalkyl, unsubstituted or substituted aryl and unsubstituted or substituted heteroaryl;
   R₃ and R₄ are independently selected from the group consisting of hydrogen, unsubstituted or substituted C₁-C₄ alkyl, unsubstituted or substituted C₃-C₇ cycloalkyl, unsubstituted or substituted aryl and unsubstituted or substituted heteroaryl or COR₁₀; wherein:
R₁₀ is independently selected from the group consisting of C₁-C₅ alkyl, unsubstituted or substituted aryl and unsubstituted or substituted heteroaryl; or,
   R₃ and R₄, taken together, form an unsubstituted or substituted 5-, 6-, 7- or 8-member ring;
   R₅ and R₆ are independently selected from the group consisting of hydrogen, unsubstituted or substituted C₁-C₁₀ alkyl and substituted or unsubstituted C₃-C₇ cycloalkyl, unsubstituted or substituted aryl and substituted or unsubstituted heteroaryl; or,
   R₅ and R₆, taken together, form a 5-, 6-, 7- or 8-member cycloalkyl fused to a group selected from the group consisting of unsubstituted or substituted aryl and unsubstituted or substituted heteroaryl.

In an aspect of this invention, R₅ is selected from the group consisting of hydrogen, methyl and cyclopropyl.

In an aspect of this invention, R₅ is substituted with 1-4 groups selected from the group consisting of hydroxy, halogen, perhaloalkyl, —OR₁₁, —NR₁₁, R₁₁ₐ, —CN, —C(X)R₁₁, —C(X)OR₁₁, —C(X)NR₁₁R₁₁ₐ, —N(R₁₁)—C(X)R₁₁ₐ, —N(R₁₁)—C(X)NR₁₁R₁₁ₐ and —OC(X)R₁₁, wherein:
X is oxygen; and,
R₁₁ and R₁₁ₐ are independently selected from the group consisting of hydrogen, C₁-C₄ alkyl, C₂-C₄ alkenyl, C₂-C₄ alkynyl, C₃-C₇ cycloalkyl, and C₅-C₁₀ cycloalkenyl.

In an aspect of this invention, R₆ is unsubstituted or substituted phenyl wherein, if substituted, the substituent is one or more selected from the group consisting of C₁-C₄ alkyl, hydroxyl, halogen, perhaloalkyl, and OR₉, wherein:
R₉ is selected from the group consisting of hydrogen, methyl, fluoro, chloro, bromo, —CN, trifluoromethyl.

In an aspect of this invention, R₆ is unsubstituted or substituted heteroaryl wherein, if substituted, the substituent is one or more independently selected from the group consisting of halogen, C₁-C₁₀alkyl and perhaloalkyl. In an aspect of this invention the heteroaryl is pyridine or furan.

In an aspect of this invention, the substituent on R₅ is selected from the group consisting of methyl, trifluoromethyl and chloro.

In an aspect of this invention, R₆ is unsubstituted or substituted C₁-C₁₀ alkyl, wherein, if substituted, the substituent is one more independently selected from the group consisting of unsubstituted C₁-C₅ alkyl and —C(O)OR₁₀, wherein R₁₀ is unsubstituted C₁-C₅ alkyl.

In an aspect of this invention, R₆ is substituted with 1-4 groups selected from the group consisting of hydroxy, halogen, perhaloalkyl, —OR₁₁, —NR₁₁ R₁₁ₐ, —CN, —C(X)R₁₁, —C(X)OR₁₁, —C(X)NR₁₁R₁₁ₐ, —N(R₁₁)—C(X)R₁₁ₐ, —N(R₁₁)—C(X)NR₁₁R₁₁ₐ and —OC(X)R₁₁, wherein:
X is oxygen; and,
R₁₁ and R₁₁ₐ are independently selected from the group consisting of hydrogen, C₁-C₄ alkyl, C₂-C₄ alkenyl, C₂-C₄ alkynyl, C₃-C₇ cycloalkyl, and C₅-C₁₀ cycloalkenyl.

In an aspect of this invention, R₆ is selected from the group consisting of hydrogen, methyl and cyclopropyl.

In an aspect of this invention, R₆ is unsubstituted or substituted phenyl wherein, if substituted, the substituent is one or more selected from the group consisting of C₁-C₄ alkyl, hydroxyl, halogen, perhaloalkyl, and OR₉, wherein:
R₉ is selected from the group consisting of hydrogen, methyl, fluoro, chloro, bromo, —CN, trifluoromethyl.

In an aspect of this invention, R₆ is unsubstituted or substituted heteroaryl wherein, if substituted, the substituent is one or more independently selected from the group consisting of halogen, C₁-C₁₀alkyl and perhaloalkyl. In an aspect of this invention the heteroaryl is pyridine or furan.

In an aspect of this invention, the substituent on R₆ is selected from the group consisting of methyl, trifluoromethyl and chloro.

In an aspect of this invention, R₆ is unsubstituted or substituted C₁-C₁₀ alkyl, wherein, if substituted, the substituent is one more independently selected from the group consisting of unsubstituted C₁-C₅ alkyl and —C(O)OR₁₀, wherein R₁₀ is unsubstituted C₁-C₅ alkyl.

In an aspect of this invention, R₁ is hydrogen.

In an aspect of this invention, R₁ₐ is hydrogen.

In an aspect of this invention, R_{1b} is hydrogen.

In an aspect of this invention, R_{1c} is hydrogen.

In an aspect of this invention, R₂ is hydrogen.

In an aspect of this invention, R₂ₐ is hydrogen.

In an aspect of this invention, R₃ is benzoyl.

In an aspect of this invention, R₄ is hydrogen.

In an aspect of this invention, X is oxygen.

In an aspect of this invention, R₁, R₁ₐ, R_{1b}, R_{1c}, R₂, R₂ₐ and R₄ are hydrogen; R₃ is benzoyl; and, X is oxygen.

In an aspect of this invention, R₅ and R₆, taken together, form a 2,3-dihydro-1H-indene ring.

An aspect of this invention is a method of screening for a compound that modulates the activity of PAR2 or a PAR2 subtype, comprising:

providing a recombinant cell comprising a nucleic acid that expresses PAR2 or a PAR2 subtype;
contacting the recombinant cell with a test compound; and,
detecting changes in the activity of the PAR2 or the PAR2 subtype in the cell.

In an aspect of this invention, the above method further comprises comparing the effect of the compound on the recombinant cell with its effect on a cell that does not contain a nucleic acid that expresses PAR2 or PAR2 subtype.

In an aspect of this invention, the nucleic acid expressing PAR2 or PAR2 subtype comprises at least 20 contiguous nucleotides which hybridizes under stringent hybridization conditions to at least 20 contiguous nucleotides of a complement of nucleic acid SEQ ID NO:1 or of a nucleic acid that encodes a polypeptide having SEQ ID NO:2.

In an aspect of this invention, the nucleic acid expressing PAR2 or PAR2 subtype comprises at least 20 contiguous nucleotides which can hybridize under stringent hybridizations conditions to a complement of at least 20 contiguous nucleotides of a nucleic acid that encodes SEQ ID NO:2.

In an aspect of this invention, the nucleic acid expressing PAR2 or PAR2 subtype comprises at least 50, at least 100, at least 150, at least 200, at least 250, at least 300, at least 350, at least 400, at least 450, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, or at least 2500, contiguous nucleotides which can hybridize under stringent hybridizations conditions to a complement of the same number of contiguous nucleotides of a nucleic acid that encodes the amino acid sequence of SEQ ID NO:2.

In an aspect of this invention, the nucleic acid encodes the polypeptide sequence of SEQ ID NO:2

An aspect of this invention is a method for treating or preventing a disease or disorder related to PAR2 activity comprising administering a therapeutically effective amount of a compound of claim 1 to a patient in need thereof.

In an aspect of this invention, the disease or disorder is selected from the group consisting of:

acute or chronic pain;
acute or chronic inflammation;
diseases or disorder of the pulmonary system;
diseases or disorders of the gastrointestinal system;
diseases or disorders of the musculoskeletal system;
diseases or disorders of the central nervous system;
diseases or disorders of the cardiovascular system;
disease or disorders of the renal system;
diseases or disorders of the hepatic system;
diseases of disorders of the eye;
diseases or disorders of the skin;
diseases or disorders of the prostrate;
diseases or disorders of the pancreas;
Sjogren's syndrome; and,
dry mouth;

In an aspect of this invention, the disease or disorder of the pulmonary system is selected from the group consisting of asthma, chronic obstructive pulmonary disease, lung cancer and pneumonitis.

In an aspect of this invention, the disease or disorder of the gastrointestinal system is selected from the group consisting of gastric ulcers, colitis, inflammatory bowel syndrome, Crohn's disease, gastric and intestinal motility, colon cancer, cancer of the stomach, and cancer of the intestine.

In an aspect of this invention, the disease or disorder of the musculoskeletal system is selected from the group consisting of rheumatoid arthritis, osteoporosis and Paget's disease.

In an aspect of this invention, the disease or disorder of the central nervous system is selected from the group consisting of Alzheimer's disease, encephalitis, meningitis, ischemia and stroke.

In an aspect of this invention, the disease or disorder of the cardiovascular system is selected from the group consisting of hypertension, atherosclerosis, angina, congestive heart failure, myocarditus and cardiac ischemia.

In an aspect of this invention, the disease or disorder of the renal system is selected from the group consisting of glomerular kidney disease, kidney cancer and renal failure.

In an aspect of this invention, the disease or disorder of the hepatic system is selected from the group consisting hepatitis and liver cancer.

In an aspect of this invention, the disease or disorder of the eye is selected from the group consisting of glaucoma, retinitis pigmentosa, cataracts, macular degeneration and dry eye.

In an aspect of this invention, the disease or disorder of the skin is selected from the group consisting of dermatitis, psoriasis, pruritis, dermatitis, eczema, seborrhea, wounds, and melanoma.

In an aspect of this invention, the disease or disorder of the pancreatic system is selected from the group consisting of pancreatitus, pancreatic cancer and diabetes.

In an aspect of this invention, the disease or disorder is dry mouth.

In an aspect of this invention, the disease or disorder is Siogren's syndrome.

In an aspect of this invention, the disease or disorder is acute or chronic pain.

In an aspect of this invention, the disease or disorder is acute or chronic inflammation.

In an aspect of this invention, the disease of disorder of the prostatic system is selected from the group consisting of benign prostatic hyperplasia and prostatic cancer.

In an aspect of this invention, the disease or disorder of the pancreatic system is selected from the group consisting of pancreatitis, diabetes and pancreatic cancer.

An aspect of this invention is a method of screening for a compound that modulates the activity of PAR2 or PAR2 subtype, comprising:

contacting PAR2 or par2-subtype with a compound of claim 1; and,
detecting changes in the activity of the PAR2 or PAR2 subtype.

An aspect of this invention is a method of screening for a compound that modulates the activity of PAR2 or PAR2 subtype, comprising:

providing a plurality of cells that express PAR2 or PAR2 subtype;
incubating the cells or a component extracted from the cells with a compound of claim 1; and detecting changes in PAR2 activity in the cell.

In an aspect of this invention, the cells over-express the PAR2 or PAR2 subtype.

In an aspect of this invention, the compound is selective for PAR2 or the PAR2 subtype.

An aspect of this invention is a compound selected from the group consisting of:
N-[2-hydrazino-2-oxo-1-(4-oxo-3,4-dihydro-1-phthalazinyl)ethyl]benzamide,
N-[2-{(2E)-2-[1-(3-bromophenyl)ethylidene]hydrazino}-2-oxo-1-(4-oxo-3,4-dihydro-1-phthalazinyl)ethyl]benzamide,
N-[2-[(2E)-2-(3-bromobenzylidene)hydrazino]-2-oxo-1-(4-oxo-3,4-dihydro-1-phthalazinyl)ethyl]benzamide,
N-[2-[(2E)-2-(2-furylmethylene)hydrazino]-2-oxo-1-(4-oxo-3,4-dihydro-1-phthalazinyl)ethyl]benzamide,
N-[2-{(2E)-2-1-(3-methoxyphenyl)ethylidene]hydrazino)-2-oxo-1-(4-oxo-3,4-dihydro-1-phthalazinyl)ethyl}benzamide,
N-[2-[(2E)-2-(2-hydroxybenzylidene)hydrazino]-2-oxo-1-(4-oxo-3,4-dihydro-1-phthalazinyl)ethyl]benzamide,
N-[2-[(2E)-2-(2-bromobenzylidene)hydrazino]-2-oxo-1-(4-oxo-3,4-dihydro-1-phthalazinyl)ethyl]benzamide,
N-[2-{(2E)-2-[1-(4-bromophenyl)ethylidene]hydrazino}-2-oxo-1-(4-oxo-3,4-dihydro-1-phthalazinyl)ethyl]benzamide,
N-[2-(2E)-2-[1-(2-methoxyphenyl)ethylidene]hydrazino)-2-oxo-1-(4-oxo-3,4-dihydro-1-phthalazinyl)ethyl]benzamide,
N-[2-[(2E)-2-(6-methoxy-2,3-dihydro-1H-inden-1-ylidene)hydrazino]-2-oxo-1-(4-oxo-3,4-dihydro-1-phthalazinyl)ethyl]benzamide,
N-[2-[(2E)-2-(4-fluorobenzylidene)hydrazino]-2-oxo-1-(4-oxo-3,4-dihydro-1-phthalazinyl)ethyl]benzamide,
N-[2-{(2E)-2-[1-(2-fluorophenyl)ethylidene]hydrazino}-2-oxo-1-(4-oxo-3,4-dihydro-1-phthalazinyl)ethyl]benzamide,
N-[2-(2E)-2-[1-(2-bromophenyl)ethylidene]hydrazino)-2-oxo-1-(4-oxo-3,4-dihydro-1-phthalazinyl)ethyl]benzamide,
N-[2-[(2E)-2-(2-fluorobenzylidene)hydrazino]-2-oxo-1-(4-oxo-3,4-dihydro-1-phthalazinyl)ethyl]benzamide,
N-(2-oxo-1-(4-oxo-3,4-dihydro-1-phthalazinyl)-2-(2E)-2-[3-(trifluoromethyl)-benzylidene]hydrazinolethyl)benzamide,
N-[2-oxo-1-(4-oxo-3,4-dihydro-1-phthalazinyl)-2-[(2E)-2-(2-pyridinylmethylene)-hydrazino]ethyl]benzamide,
N-2-oxo-1-(4-oxo-3,4-dihydro-1-phthalazinyl)-2-[(2E)-2-(3-pyridinylmethylene)-hydrazino]ethylbenzamide,
N-[2-((2E)-2-[3-chloro-5-(trifluoromethyl)-2-pyridinyl]methylene)-hydrazino)-2-oxo-1-(4-oxo-3,4-dihydro-1-phthalazinyl)ethyl]benzamide,
N-(2-oxo-1-(4-oxo-3,4-dihydro-1-phthalazinyl)-2-(2E)-2-[1-(3-pyridinyl)-ethylidene]hydrazino)ethyl)benzamide,
N-(2-oxo-1-(4-oxo-3,4-dihydro-1-phthalazinyl)-2-{(2E)-2-[1-(2-pyridinyl)ethylidene]hydrazino}ethyl)benzamide,
N-[2-[(2E)-2-(1,3-dimethylbutylidene)hydrazino]-2-oxo-1-(4-oxo-3,4-dihydro-1-phthalazinyl)ethyl]benzamide,
N-[2-{(2E)-2-[2-(3-methoxyphenyl)-1-methylethylidene]hydrazino}-2-oxo-1-(4-oxo-3,4-dihydro-1-phthalazinyl)ethyl]benzamide,
N-[2-{(2E)-2-[1-(3-bromophenyl)propylidene]hydrazino}-2-oxo-1-(4-oxo-3,4-dihydro-1-phthalazinyl)ethyl]benzamide,
N-[2-{(2E)-2-[1-(3-fluorophenyl)ethylidene]hydrazino}-2-oxo-1-(4-oxo-3,4-dihydro-1-phthalazinyl)ethyl]benzamide,
N-[2-oxo-1-(4-oxo-3,4-dihydro-1-phthalazinyl)-2-((2E)-2-{1-[3-(trifluoromethyl)phenyl]ethylidene}hydrazino)ethyl]benzamide,
N-[2-{(2E)-2-[1-(3-chlorophenyl)ethylidene]hydrazino}-2-oxo-1-(4-oxo-3,4-dihydro-1-phthalazinyl)ethyl]benzamide,
N-[2-{(2E)-2-[1-(3-cyanophenyl)ethylidene]hydrazino}-2-oxo-1-(4-oxo-3,4-dihydro-1-phthalazinyl)ethyl]benzamide,
N-[2-{(2E)-2-[1-(3-hydroxyphenyl)ethylidene]hydrazino}-2-oxo-1-(4-oxo-3,4-dihydro-1-phthalazinyl)ethyl]benzamide,
N-[2-{(2E)-2-[1-(3-methylphenyl)ethylidene]hydrazino}-2-oxo-1-(4-oxo-3,4-dihydro-1-phthalazinyl)ethyl]benzamide,
N-[2-{(2E)-2-[1-(4-methyl-2-pyridinyl)ethylidene]hydrazino}-2-oxo-1-(4-oxo-3,4-dihydro-1-phthalazinyl)ethyl]benzamide,
N-[2-{(2E)-2-[1-(3-bromophenyl)-2,2-dimethylpropylidene]-hydrazino}-2-oxo-1-(4-oxo-3,4-dihydro-1-phthalazinyl)ethyl]benzamide,
N-[2-{(2E)-2-[1-(3-bromophenyl)-2-methylpropylidene]hydrazino}-2-oxo-1-(4-oxo-3,4-dihydro-1-phthalazinyl)ethyl]benzamide,
N-[2-{(2E)-2-[(3-bromophenyl)(cyclopropyl)methylene]hydrazino}-2-oxo-1-(4-oxo-3,4-dihydro-1-phthalazinyl)ethyl]benzamide,
N-[2-{(2E)-2-[1-(3-bromophenyl)butylidene]hydrazino}-2-oxo-1-(4-oxo-3,4-dihydro-1-phthalazinyl)ethyl]benzamide,
N-[2-[(2E)-2-(2,2-dimethyl-1-phenylpropylidene)hydrazino]-2-oxo-(4-oxo-3,4-dihydro-1-phthalazinyl)ethyl]benzamide,
N-[2-{(2E)-2-[1-(3,5-dimethoxyphenyl)ethylidene]hydrazino}-2-oxo-1-(4-oxo-3,4-dihydro-1-phthalazinyl)ethyl]benzamide,
N-[2-((2E)-2-{1-[3,5-bis(trifluoromethyl)phenyl]ethylidene}-hydrazino)-2-oxo-1-(4-oxo-3,4-dihydro-1-phthalazinyl)ethyl]benzamide,
N-[2-{(2E)-2-[1-(3,5-difluorophenyl)ethylidene]hydrazino}-2-oxo-1-(4-oxo-3,4-dihydro-1-phthalazinyl)ethyl]benzamide,
N-[2-[(2E)-2-(3,5-dibromobenzylidene)hydrazino]-2-oxo-1-(4-oxo-3,4-dihydro-1-phthalazinyl)ethyl]benzamide,
N-[2-{(2E)-2-[1-(3,5-dibromophenyl)ethylidene]hydrazino}-2-oxo-1-(4-oxo-3,4-dihydro-1-phthalazinyl)ethyl]benzamide, and,
N-[2-((2E)-2-[1-(3,5-dimethylphenyl)ethylidene]hydrazino)-2-oxo-1-(4-oxo-3,4-dihydro-1-phthalazinyl)ethyl]benzamide.

An aspect of this invention is a method for treating or preventing a disease or disorder related to PAR2 activity comprising administering a therapeutically effective amount to a patient in need thereof of a compound selected from the group consisting of:
N-[2-hydrazino-2-oxo-1-(4-oxo-3,4-dihydro-1-phthalazinyl)ethyl]benzamide,
N-[2-{(2E)-2-[1-(3-bromophenyl)ethylidene]hydrazino}-2-oxo-1-(4-oxo-3,4-dihydro-1-phthalazinyl)ethyl]benzamide,
N-[2-[(2E)-2-(3-bromobenzylidene)hydrazino]-2-oxo-1-(4-oxo-3,4-dihydro-1-phthalazinyl)ethyl]benzamide,
N-[2-[(2E)-2-(2-furylmethylene)hydrazino]-2-oxo-1-(4-oxo-3,4-dihydro-1-phthalazinyl)ethyl]benzamide,
N-[2-{(2E)-2-[1-(3-methoxyphenyl)ethylidene]hydrazino}-2-oxo-1-(4-oxo-3,4-dihydro-1-phthalazinyl)ethyl]benzamide,
N-[2-[(2E)-2-(2-hydroxybenzylidene)hydrazino]-2-oxo-1-(4-oxo-3,4-dihydro-1-phthalazinyl)ethyl]benzamide,
N-[2-[(2E)-2-(2-bromobenzylidene)hydrazino]-2-oxo-1-(4-oxo-3,4-dihydro-1-phthalazinyl)ethyl]benzamide,
N-[2-((2E)-2-[1-(4-bromophenyl)ethylidene]hydrazino)-2-oxo-1-(4-oxo-3,4-dihydro-1-phthalazinyl)ethyl]benzamide,
N-[2-{(2E)-2-[1-(2-methoxyphenyl)ethylidene]hydrazino}-2-oxo-1-(4-oxo-3,4-dihydro-1-phthalazinyl)ethyl]benzamide,
N-[2-[(2E)-2-(6-methoxy-2,3-dihydro-1H-inden-1-ylidene)hydrazino]-2-oxo-1-(4-oxo-3,4-dihydro-1-phthalazinyl)ethyl]benzamide,
N-[2-[(2E)-2-(4-fluorobenzylidene)hydrazino]-2-oxo-1-(4-oxo-3,4-dihydro-1-phthalazinyl)ethyl]benzamide,
N-[2-{(2E)-2-[1-(2-fluorophenyl)ethylidene]hydrazino}-2-oxo-1-(4-oxo-3,4-dihydro-1-phthalazinyl)ethyl]benzamide,
N-[2-{(2E)-2-[1-(2-bromophenyl)ethylidene]hydrazino}-2-oxo-1-(4-oxo-3,4-dihydro-1-phthalazinyl)ethyl]benzamide,
N-[2-[(2E)-2-(2-fluorobenzylidene)hydrazino]-2-oxo-1-(4-oxo-3,4-dihydro-1-phthalazinyl)ethyl]benzamide,
N-{2-oxo-1-(4-oxo-3,4-dihydro-1-phthalazinyl)-2-((2E)-2-[3-(trifluoromethyl)-benzylidene]hydrazino}ethyl)benzamide,
N-{2-oxo-1-(4-oxo-3,4-dihydro-1-phthalazinyl)-2-[(2E)-2-(2-pyridinylmethylene)-hydrazino]ethyl}benzamide,
N-{2-oxo-1-(4-oxo-3,4-dihydro-1-phthalazinyl)-2-[(2E)-2-(3-pyridinylmethylene)-hydrazino]ethyl}benzamide,
N-[2-((2E)-2-{[3-chloro-5-(trifluoromethyl)-2-pyridinyl]methylene}-hydrazino)-2-oxo-1-(4-oxo-3,4-dihydro-1-phthalazinyl)ethyl]benzamide,
N-(2-oxo-1-(4-oxo-3,4-dihydro-1-phthalazinyl)-2-{(2E)-2-[1-(3-pyridinyl)-ethylidene]hydrazino}ethyl)benzamide,
N-(2-oxo-1-(4-oxo-3,4-dihydro-1-phthalazinyl)-2-{(2E)-2-[1-(2-pyridinyl)ethylidene]hydrazino}ethyl)benzamide,
N-[2-[(2E)-2-(1,3-dimethylbutylidene)hydrazino]-2-oxo-1-(4-oxo-3,4-dihydro-1-phthalazinyl)ethyl]benzamide,
N-[2-{(2E)-2-[2-(3-methoxyphenyl)-1-methylethylidene]hydrazino}-2-oxo-1-(4-oxo-3,4-dihydro-1-phthalazinyl)ethyl]benzamide,
N-[2-{(2E)-2-[1-(3-bromophenyl)propylidene]hydrazino}-2-oxo-1-(4-oxo-3,4-dihydro-1-phthalazinyl)ethyl]benzamide,
N-[2-{(2E)-2-[1-(3-fluorophenyl)ethylidene]hydrazino}-2-oxo-1-(4-oxo-3,4-dihydro-1-phthalazinyl)ethyl]benzamide,
N-[2-oxo-1-(4-oxo-3,4-dihydro-1-phthalazinyl)-2-((2E)-2-{1-[3-(trifluoromethyl)phenyl]ethylidene}hydrazino)ethyl]benzamide,
N-[2-{(2E)-2-[1-(3-chlorophenyl)ethylidene]hydrazino}-2-oxo-1-(4-oxo-3,4-dihydro-1-phthalazinyl)ethyl]benzamide,
N-[2-{(2E)-2-[1-(3-cyanophenyl)ethylidene]hydrazino}-2-oxo-1-(4-oxo-3,4-dihydro-1-phthalazinyl)ethyl]benzamide,
N-[2-{(2E)-2-[1-(3-hydroxyphenyl)ethylidene]hydrazino}-2-oxo-1-(4-oxo-3,4-dihydro-1-phthalazinyl)ethyl]benzamide,
N-[2-{(2E)-2-[1-(3-methylphenyl)ethylidene]hydrazino}-2-oxo-1-(4-oxo-3,4-dihydro-1-phthalazinyl)ethyl]benzamide,
N-[2-{(2E)-2-[1-(4-methyl-2-pyridinyl)ethylidene]hydrazino}-2-oxo-1-(4-oxo-3,4-dihydro-1-phthalazinyl)ethyl]benzamide,
N-[2-{(2E)-2-[1-(3-bromophenyl)-2,2-dimethylpropylidene]-hydrazino}-2-oxo-1-(4-oxo-3,4-dihydro-1-phthalazinyl)ethyl]benzamide,
N-[2-{(2E)-2-[1-(3-bromophenyl)-2-methylpropylidene]hydrazino}-2-oxo-1-(4-oxo-3,4-dihydro-1-phthalazinyl)ethyl]benzamide,
N-[2-((2E)-2-[(3-bromophenyl)(cyclopropyl)methylene]hydrazino)-2-oxo-1-(4-oxo-3,4-dihydro-1-phthalazinyl)ethyl]benzamide,
N-[2-{(2E)-2-[1-(3-bromophenyl)butylidene]hydrazino}-2-oxo-1-(4-oxo-3,4-dihydro-1-phthalazinyl)ethyl]benzamide,
N-[2-[(2E)-2-(2,2-dimethyl-1-phenylpropylidene)hydrazino]-2-oxo-(4-oxo-3,4-dihydro-1-phthalazinyl)ethyl]benzamide,
N-[2-{(2E)-2-[1-(3,5-dimethoxyphenyl)ethylidene]hydrazino}-2-oxo-1-(4-oxo-3,4-dihydro-1-phthalazinyl)ethyl]benzamide,
N-[2-((2E)-2-{1-[3,5-bis(trifluoromethyl)phenyl]ethylidene}-hydrazino)-2-oxo-1-(4-oxo-3,4-dihydro-1-phthalazinyl)ethyl]benzamide,
N-[2-((2E)-2-[1-(3,5-difluorophenyl)ethylidene]hydrazino)-2-oxo-1-(4-oxo-3,4-dihydro-1-phthalazinyl)ethyl]benzamide,
N-[2-[(2E)-2-(3,5-dibromobenzylidene)hydrazino]-2-oxo-1-(4-oxo-3,4-dihydro-1-phthalazinyl)ethyl]benzamide,
N-[2-{(2E)-2-[1-(3,5-dibromophenyl)ethylidene]hydrazino}-2-oxo-1-(4-oxo-3,4-dihydro-1-phthalazinyl)ethyl]benzamide; and,
N-[2-{(2E)-2-[1-(3,5-dimethylphenyl)ethylidene]hydrazino}-2-oxo-1-(4-oxo-3,4-dihydro-1-phthalazinyl)ethyl]benzamide.

### DETAILED DESCRIPTION

### Brief Description of the Figures

FIG. 1 is a graph of the PAR2 activating ability of several compounds herein compared to SLIGRL. The abscissa of the graph is calibrated in Absorbance Units and the Ordinate in ligand concentration.

FIG. 2 is a series of graphs showing the selectivity of several compounds herein for activation of PAR2 compared to PAR1 and PAR3/4. The abscissas and ordinates of the graph are the same as in FIG. 1.

FIG. 3 is a graph comparing the ability of several compounds herein to act at human PAR2 to stimulate intracellular calcium mobilization.

FIG. 4 is a graph comparing the ability of several compounds herein to act at human PAR2 to stimulate inositol phosphate hydrolysis. The abscissa is calibrated in Disintegrations Per Minute and the ordinate in compound concentration.

### DEFINITIONS

As used in these definitions, any “R” group(s), such as, without limitation, R, R^{a} and R^{b}, is(are) independently selected from the group consisting of hydrogen, alkyl, alkenyl, alkynyl, cycloalkyl, aryl, heteroaryl (bonded to the indicated group at a ring carbon atom) and heteroalicyclyl (likewise bonded to the indicated group at a ring carbon atom), as these groups are defined herein. If two “R” groups are covalently bonded to the same atom or to adjacent atoms, then they may be “taken together” as defined herein to form a cycloalkyl, aryl, heteroaryl or heteroalicyclyl group.

Whenever a group of this invention is described as being “optionally substituted” that group may be unsubstituted or substituted with one or more of the indicated substituents. Likewise, when a group is described as being “unsubstituted or substituted,” if substituted, the substituent may be selected from the same group of substituents.

As used herein, “Cₘ to Cₙ” in which “m” and “n” are integers refers to the number of carbon atoms in an alkyl, alkenyl or alkynyl group. That is, the alkyl, alkenyl or alkynyl can contain from “m” to “n”, inclusive, carbon atoms. If no “m” and “n” are designated with regard to an alkyl, alkenyl or alkynyl group herein, the broadest range described in these definitions is to be assumed. Thus “alkyl” alone means C₁-C₂₀ alkyl. A “C₁ to C₄ alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)— and (CH₃)₃CH—, etc. With regard to cyclic compounds, “m” and “n” provide the number of possible carbon atoms in the ring.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene.

As used herein, “heteroaryl” refers to a ring or two or more fused rings that contain(s) one or more heteroatoms selected from the group consisting of nitrogen, oxygen and sulfur in the ring and that have a fully delocalized pi-electron system. Examples of heteroaryl rings include, but are not limited to, furan, thiophene, phthalazinone, pyrrole, oxazole, thiazole, imidazole, imidazoline, imidazolidine, pyrazole, isoxazole, isothiazole, triazole, thiadiazole, pyran, pyridine, pyridazine, pyrimidine, pyrazine, triazine,

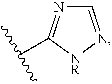

and

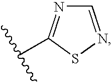

with R as defined herein.

As used herein, “alkyl” refers to a straight or branched chain fully saturated (no double or triple bonds) hydrocarbon group. An alkyl group of this invention may comprise from 1 to 20 carbon atoms, that is, m=1 and n=20. An alkyl group herein may also be of medium size having 1 to 10 carbon atoms. It is presently preferred that an alkyl group of this invention be a lower alkyl having 1 to 5 carbon atoms. Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

An alkyl group of this invention may be substituted or unsubstituted. When substituted, the substituent group(s) is(are) one or more group(s) independently selected from cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR^{a}R^{b} and protected amino.

As used herein, “perfluoroalkyl” refers to an alkyl group as defined herein wherein all of the hydrogen atoms attached to carbon are substituted with a chlorine or fluorine.

As used herein, “alkenyl” refers to an alkyl group that contains in the straight or branched hydrocarbon chain one or more double bonds. An alkenyl group of this invention may be unsubstituted or substituted. When substituted, the substituent(s) may be selected from the same groups disclosed above with regard to alkyl group substitution.

As used herein, “alkynyl” refers to an alkyl group that contains in the straight or branched hydrocarbon chain one or more triple bonds. An alkynyl group of this invention may be unsubstituted or substituted. When substituted, the substituent(s) may be selected from the same groups disclosed above with regard to alkyl group substitution.

As used herein, “acyl” refers to an “RC(═O)O—” group.

As used herein, “cycloalkyl” refers to a completely saturated (no double bonds) hydrocarbon ring. Cycloalkyl groups of this invention may range from C₃ to C₈. A cycloalkyl group may be unsubstituted or substituted. If substituted, the substituent(s) may be selected from those indicated above with regard to substitution of an alkyl group.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). An cycloalkenyl group of this invention may unsubstituted or substituted. When substituted, the substituent(s) may be selected from the same groups disclosed above with regard to alkyl group substitution.

As used herein, “heteroalicyclic” or heteroalicyclyl” refers to a ring or one or more fused rings having in the ring system one or more heteroatoms independently selected from nitrogen, oxygen and sulfur. The rings may also contain one or more double bonds provided that they do not create a fully delocalized pi-electron system in the rings. Heteroalicyclyl groups of this invention may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethanesulfonamido.

An “O-carboxy” group refers to a “RC(═O)O—” group with R as defined above.

A “C-carboxy” group refers to a “—C(═O)OR” group with R as defined above.

An “acetyl” group refers to a CH₃C(═O)— group.

A “trihalomethanesulfonyl” group refers to an “X₃CSO₂—” group wherein X is a halogen.

A “cyano” group refers to a “—CN” group.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group with R as defined above.

An “S-sulfonamido” group refers to a “—SO₂NR” group with R as defined above.

An “N-sulfonamido” group refers to a “RSO₂NH—” group with R as defined above.

A “trihalomethanesulfonamido” group refers to an “X₃CSO₂NR—” group with X as halogen and R as defined above.

An “O-carbamyl” group refers to a “—OC(═O)—NR” group with R as defined above.

An “N-carbamyl” group refers to an “ROC(═O)NR^{a}—”, group with R^{a} and R as defined above.

An “O-thiocarbamyl” group refers to a “—OC(═S)—NR^{a}R^{b}” group with R^{a} and R^{b} as defined above.

An “N-thiocarbamyl” group refers to an “ROC(═S)NR^{a}—”, group with R^{a} and R as defined above.

A “C-amido” group refers to a “—C(═O)—NR^{a}R^{b}” group with R^{a} and R^{b} as defined above.

An “N-amido” group refers to a RC(═O)NR^{a}— group with R as defined above.

The term “perhaloalkyl” refers to an alkyl group in which all the hydrogen atoms are replaced by halogen atoms.

As used herein, an “ester” refers to a “—C(O)OR^{a}” group with R^{a} as defined above.

As used herein, an “amide” refers to a “—C(O)NR^{a}R^{b}” group with R^{a} and R^{b} as defined above.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999).

As used herein, the phrase “taken together” when referring to two “R” groups means that the “R” groups are joined together to form a cycloalkyl, aryl, heteroaryl or heteroalicyclyl group. For example, without limitation, if R^{a} and R^{b} of an NR^{a}R^{b} group are indicated to be “taken together,” it means that they are covalently bonded to one another at their terminal atoms to form a ring:

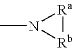

It is understood that, in any compound of this invention having one or more chiral centers, if an absolute stereochemistry is not expressly indicated, then each center may independently be R or S or a mixture thereof.

As used herein, “stringent hybridization conditions” refers to

As used herein, “pharmaceutically acceptable salt” refers to a salt of a compound that does not cause significant irritation to a patient to which it is administered and does not abrogate the biological activity and properties of the compound. Pharmaceutical salts can be obtained by reaction of a compound disclosed herein with an acid or base to form a salt. Basic salts include, without limitation, ammonium salt (NH₄⁺) salts; alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of with amino acids such as, without limitation, arginine and lysine. Useful acid salts include, without limitation, hydrochlorides, hydrobromides, sulfates, nitrates, phosphates, methanesulfonates, ethanesulfonates, p-toluenesulfonates and salicylates.

As used herein, a “prodrug” refers to a compound that may not be pharmaceutically active but that is converted into an active drug in vivo. Prodrugs are often useful because they may be easier to administer than the parent drug. They may, for example, be bioavailable by oral administration whereas the parent drug is not. The prodrug may also have better solubility than the active parent drug in pharmaceutical compositions. An example, without limitation, of a prodrug would be a compound disclosed herein, which is administered as an ester (the “prodrug”) to facilitate absorption through a cell membrane where water solubility is detrimental to mobility but which then is metabolically hydrolyzed to a carboxylic acid (the active entity) once inside the cell where water-solubility is beneficial. A further example of a prodrug might be a short peptide (polyaminoacid) bonded to an acid group where the peptide is metabolized in vivo to reveal the active parent.

As used herein, the phrase “a disease or disorder related to PAR2 activity” means that the pathogenesis of the disease or disorder involves PAR2 activity, either over-activity, normal activity or underactivity and it is know or believed that a change in the activity level of PAR2 would or might have a beneficial effect, that is, would or might ameliorate or cure the disease or disorder.

As used herein, to “modulate” the activity of PAR2 means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all even in the presence of a natural binding partner. A natural binding partner is an endogenous molecule that is an agonist for the receptor.

As used herein, to “detect” changes in the activity of PAR2 or of a PAR2 sub-type refers to the process of analyzing the result of an experiment using whatever analytical techniques are best suited to the particular situation. In some cases simple visual observation may suffice, in other cases the use of a microscope, visual or UV light analyzer or specific protein assays may be required. The proper selection of analytical tools and techniques to detect changes in the activity of PAR2 or a PAR2 sub-type are well-known to those skilled in the art.

As used herein, an “agonist” refers to a compound that binds to a receptor to from a complex that elicits the full pharmacological response associated with that particular receptor. That is, an agonist for PAR2 may elicit, without limitation, the following responses: mobilization of intracellular calcium, stimulation of phosphatidyl inositol turnover or stimulation of cellular proliferation.

As used herein, “partial agonist” refers to a compound that has an affinity for a receptor but, unlike a full agonist, when bound to the receptor it elicits only a small degree of the pharmacological response normally associated with the receptor even if a large fraction of receptors are occupied by the compound.

As used herein, “inverse agonist” refers to a compound that inhibits the constitutive activity of a receptor such that the compound is not technically an antagonist but, rather, is an agonist with negative instrinsic activity.

As used herein, “antagonist” refers to a compound that binds to a receptor to form a complex that does not give rise to any response, as if the receptor were unoccupied. An antagonist may bind reversibly or irreversibly to the receptor, effectively eliminating the activity of the receptor permanently or at least until the antagonist is metabolized, dissociates, or otherwise removed by biological process.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes; and, in particular, humans.

As used herein, a “patient” refers to a subject that is being treated by an M.D. or a D.V.M. to attempt to cure, or at least ameliorate the effects of, a particular disease or disorder or to prevent the disease or disorder from occurring in the first place.

As used herein, a “therapeutically effective amount” refers to an amount of a compound that elicits the desired biological or medicinal response in an subject.

As used herein, a “pharmaceutical composition” refers to a mixture of a compound of this invention with other chemical components such as diluents, carriers or other excipients. A pharmaceutical composition may facilitate administration of the compound to a subject. Many techniques of administering a compound exist are known in the art, such as, without limitation, orally, intramuscularly, intraocularly, intranasally, parenterally, intravenously and topically. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

### Discussion

### Synthesis

A general synthetic route to the compounds of this invention is shown in Scheme 1. The route shown is illustrative only and is not intended, nor is it to be construed, to limit the scope of this invention in any manner whatsoever. Those skilled in the art will be able to recognize modifications of the disclosed synthesis and/or to devise alternate routes. All such modifications and alternate routes are within the scope of this invention.

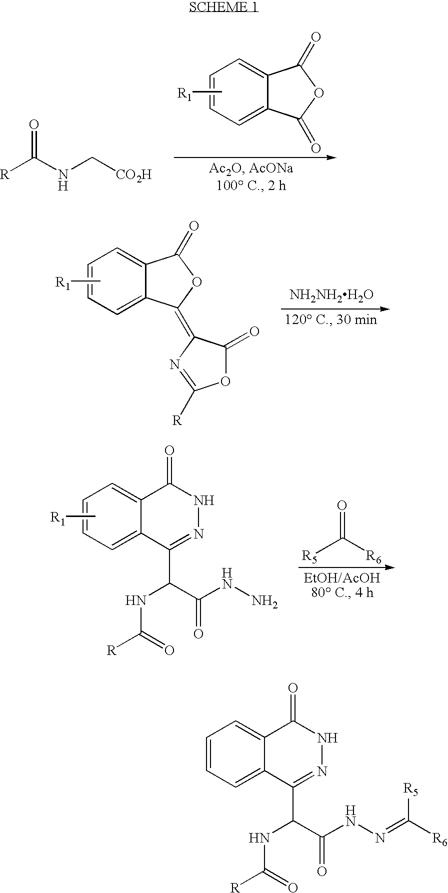

### Utility of PAR2 and Compounds Modulating its Activity

Disclosed herein is the use of PAR2 or a PAR2 subtype as a screening tool to identify compounds effective in treating or preventing diseases and disorders including, but not limited to diseases and disorders of the lung such as asthma, chronic obstructive pulmonary disease, lung cancer and pneumonitus; diseases and disorders of the stomach, small intestine, and large intestine such as gastric ulcers, colitis, inflammatory bowel syndrome, Crohn's disease, gastric and intestinal motility, colon cancer, cancer of the stomach, and cancer of the intestine; diseases and disorders of the joints such as rheumatoid arthritis; diseases and disorders of the central nervous system such as Alzheimer's disease, encephalitis, meningitis, ischemia and stroke; diseases and disorders of the skin such as dermatitis, psoriasis, pruritis, dermatitis, eczema, seborrhea, wounds, and melanoma; diseases and disorders of the cardiovascular system such as hypertension, atherosclerosis, angina, congestive heart failure, myocarditis and cardiac ischemia; diseases and disorders of the renal (kidney) system such as glomerular kidney disease, kidney cancer and renal failure; diseases and disorders of the hepatic (liver) system such as hepatitis and liver cancer; disease and disorders of the prostatic system such as benign prostatic hyperplasia and prostate cancer; diseases and disorders of the pancreas such as pancreatitis, pancreatic cancer and diabetes; diseases and disorders of the eye such as glaucoma, retinitis pigmentosa, cataracts and macular degeneration; diseases and disorders of the musculoskeletal system such as osteoporosis and Paget's disease; acute and chronic pain, acute and chronic inflammation; dry eye; dry mouth and Sjogren's syndrome. The use of PAR2 or a PAR2 subtype may comprise: a) contacting a recombinant cell with a test compound, where the recombinant cell comprises a recombinant nucleic acid expressing PAR2, provided that the cell does not have functional PAR2 expression from endogenous nucleic acid, and b) determining the ability of the test compound to affect one or more activities of PAR2, and comparing that ability with the ability of the test compound to affect the one or more PAR2 activities in a cell not comprising the recombinant nucleic acid; where the recombinant nucleic acid comprises a PAR2 nucleic acid selected from the group consisting of: i) nucleic acid of SEQ ID NO:1, ii) nucleic acid encoding the amino acid SEQ ID NO:2, iii) a derivative of either nucleic acid molecule in i) or ii), where the derivative encodes a receptor having one or more activities of PAR2 and comprises at least 20 contiguous nucleotides which can hybridize under stringent hybridization conditions to the complement of the nucleic acid of SEQ ID NO:1.

The PAR2 nucleic acid of this invention encodes the amino acid sequence of a SEQ ID NO:2 derivative comprising at least 20 contiguous nucleotides which can hybridize under stringent conditions to a complement of at least 20 contiguous nucleotides encoding the amino acid sequence of SEQ ID NO:2.

The above derivative can alternatively comprise at least 50, at least 100, at least 150, at least 200, at least 250, at least 300, at least 350, at least 400, at least 450, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, or at least 2500, contiguous nucleotides which can hybridize under stringent hybridizations conditions to a complement of contiguous nucleotides encoding the amino acid sequence of SEQ ID NO:2.

The compounds of this invention may be used to treat acute and chronic inflammation of any type by administering to a patient an effective amount of at least one compound of this invention, wherein the compound activates a PAR2 subtype.

Likewise, the compounds of this invention may be used to treat or prevent inflammation by administering to a patient suffering from inflammation an effective amount of at least one compound of this invention, whereby one or more symptoms of the inflammation is reduced.

Of course, as an aspect of this invention, a patient in need of inflammatory treatment needs to be identified as such prior to administration of the compound(s) of this invention.

The compounds of this invention preferably selectively modulate PAR2 or a PAR2 subtype, more preferably at present by activating it, i.e., by being an agonist thereof.

Inflammation or an inflammatory response may be treated in a patient by administering to the patient an effective anti-inflammatory amount of a compound of this invention. The inflammatory response may result, without limitation, from the activation of leukocytes, which activation comprises leukocyte migration and generation of reactive oxygen species to evoke vascular leakage or edema. The inflammatory response may, in an alternative, result from activation of blood monocytes and neutrophils that infiltrate the affected tissue or organ and in turn activate inflammatory mediators. Or it may be associated with rheumatoid arthritis, Alzheimer's disease or asthma. It may be associated with pulmonary disorders such as chronic obstructive pulmonary disease or asthma. The inflammatory response may likewise be associated with gastric ulcers. It may be associated with colitis and inflammatory bowel syndrome. It may be associated with pancreatitus. The inflammatory response may be associated with hepatitis. It may be associated with encephalitis. Or it may be associated with dermatitis. The inflammatory response may result from physical injury such as, without limitation, physical trauma and radiation exposure.

Vasodilation may be induced to treat or prevent a vasocontractive response or condition by administering to a patient a vasodilatory effective amount of a compound of this invention. The vasocontractive response or condition may be related to a renal hemodynamic disease, including glomerular disease or a cardiovascular disease such as, without limitation, hypertension, congestive heart failure, atherosclerosis, myocarditis, myocardial infarction, and myocardial ischemia.

On the other hand, a vasoconstrictive response may be reduced or eliminated in a patient by administering to the patient a compound of this invention. The vasoconstrictive response may be associated with a medical disorder such as, without limitation, asthma, anaphylactic shock, allergic reactions, inflammation, rheumatoid arthritis, gout, psoriasis, allergic rhinitis, adult respiratory distress syndrome, Crohn's disease, endotoxin shock, traumatic shock, hemorrhagic shock, bowel ischemic shock, renal glomerular disease, benign prostatic hypertrophy, inflammatory bowel disease, myocardial ischemia, myocardial infarction, circulatory shock, brain injury, systemic lupus erythematosus, chronic renal disease, cardiovascular disease, and hypertension. Or the vasoconstrictive response may be a renal vasoconstrictive response such as, without limitation, the response associated with chronic renal disease or glomerular kidney disease.

Acute or chronic pain may be treated or prevented by administering to a patient an effective amount of a compound or compounds of this invention.

Diseases of the eye such as, without limitation, glaucoma, cataracts and macular degeneration may be treated using a compound of this invention as can dry eye.

Dry mouth caused by, without limitation, disease or as a side effect of medications or a disorder such as, without limitation, Sjogren's syndrome may also be treated or prevented by administration of a therapeutically effective amount of a compound or compounds of this invention to a patient in need thereof.

Diseases of the bone, such as osteoporosis and Paget's disease, may also be treated or prevented by administration of a therapeutically effective amount of a compound or compounds of this invention to a patient in need thereof.

Acute and chronic pain may also be treated or prevented by administering to a patient an effective amount of at least one compound of this invention, whereby one or more symptoms of the pain are reduced.

The compounds of this invention may be selective for PAR2 or a PAR2 subtype; that is, they bind only to PAR2 (or the subtype) such that their therapeutic effects are directly related to modulation of PAR2 (or PAR2 subtype) activity.

A method of identifying a compound that regulates the activity of PAR2 may comprise, under this invention, contacting PAR2 with a compound of this invention and detecting any change in the activity level of the PAR2.

Further, a method of identifying a compound which regulates activity of the PAR2 may comprise, under this invention, culturing cells that express PAR2; incubating the cells with a compound of this invention and detecting any change in the activity of PAR2. If desired, the cultured cells may be engineered to over-express PAR2.

The compounds of this invention may be specific agonists, partial agonists, inverse agonists and/or antagonists of PAR2, thus affecting biological processes involving PAR2 and thereby being useful to further elucidate the manner of participation of PAR2 in those biological processes.

### Pharmaceutics

The compounds of this invention can be administered to a human patient per se, or in a pharmaceutical composition where they are mixed with other active ingredients as, for example, in a combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, without limitation, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternatively, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured procedures well-known in the art, e.g., by means of conventional mixing, dissolving, granulating, dragee-making, levigating, emulsifying, encapsulating, entrapping or tabletting processes.

Pharmaceutical compositions for use in accordance with the present disclosure thus may be formulated in conventional manner using one or more pharmaceutically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic and succinic. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve their intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition (Fingl et al. 1975, in “The Pharmacological Basis of Therapeutics”). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXAMPLES

The examples that follow are provided by way of illustration only and are not intended, nor are they to be construed, as limiting the scope of this invention in any manner whatsoever.

Among the apparatuses and procedures used to analyze the products of the syntheses exemplified below was a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammoniumacetate in water, buffer B: 10 mM ammoniumacetate in acetonitrile/water 95/5. A gradient was run from 10% B to 100% B in 10 min, stay at 100% B for 1 min, re-equilibrate for 6 min. or from 30% B to 100% B in 7 min, stay at 100% B for 1 min, re-equilibrate for 5.5 min. System was operated at 1 ml/min.

### Example 1

### (4E)-4-(3-oxo-2-benzofuran-1(3H)-ylidene)-2-phenyl-1,3-oxazol-5(4H)-one (1)

Phthalic anhydride (10.4 g, 70 mmol), hippuric acid (12.5 g, 70 mmol) and sodium acetate (5.7 g, 70 mmol) were taken up in 70 mL acetic anhydride in a 250 mL round-bottomed flask equipped with a reflux condenser and held at 100° C. for 2 h. The mixture was then cooled to 75° C., before adding 20 mL of warm water. The emulsion that formed solidified upon several washes with warm water, which was decanted between washes. When the washing water stayed colorless the solid was filtered and washed with acetone. Crude yield: 11.4 g.

### Example 2

### N-[2-hydrazino-2-oxo-1-(4-oxo-3,4-dihydro-1-phthalazinyl)ethyl]benzamide (2)

Hydrazine (35 mL) was cooled to 0° C. in an icebath before slowly adding (4E)-4-(3-oxo-2-benzofuran-1(3H)-ylidene)-2-phenyl-1,3-oxazol-5(4H)-one. The reaction mixture was held at 110° C. for 30 min, and then cooled to rt. The white solid was filtered and washed with ethanol and hot ethylene glycol in order to wash out all excess hydrazine. Yield 6.2 g.

¹H NMR (400 MHz, DMSO) δ 9.04 (d, J=7.2 Hz, 1H), 8.27 (d, J=7.6 Hz, 1H), 7.92-7.83 (m, 4H), 7.50 (d, J=6.0 Hz, 1H), 7.43 (t, J=6.8 Hz, 2H), 6.23 (d, J=6.8 Hz, 1H)

¹³C NMR (100 MHz, DMSO), δ 168.2, 166.9, 160.2, 143.1, 134.4, 134.2, 132.3, 129.7, 128.9, 128.5, 128.3, 128.0, 126.8, 125.0, 53.6

LC-MS (AP1): purity (UV/MS): 100/86, Rₜ 6.68 min

### Example 3

### N-[2-{(2E)-2-[1-(3-bromophenyl)ethylidene]hydrazino}-2-oxo-1-(4-oxo-3,4-dihydro-1-phthalazinyl)ethyl]benzamide (3)

3-Bromoacetophenone (3.18 g, 1.6 mmol) and N-[2-hydrazino-2-oxo-1-(4-oxo-3,4-dihydro-1-phthalazinyl)ethyl]benzamide were taken up in ethanol (16 mL) and acetic acid (1.6 mL) and held at 80° C. for 16 h. After cooling to rt, the precipitate was filtered and washed with ethanol, then dried under vacuum. Yield: 1.8 g.

¹H NMR (400 MHz, DMSO) δ 12.65 (s, 1H), 11.00 (s, 1H), 9.20 (d, J=8.4 Hz, 1H), 8.28 (d, J=7.6 Hz, 1H), 8.00-7.86 (m, 3H), 7.53 (t, J=7.2 Hz, 1H), 7.44 (t, J=8.0 Hz, 1H), 7.35-7.33 (m, 1H), 7.25-7.23 (m, 2H), 7.00 (t, J=7.2 Hz, 1H), 6.97 (t, J=7.2 Hz, 1H), 2.18 (s, 3H)

¹³ C NMR (100 MHz, DMSO), δ 171.2, 167.1, 146.4, 143.2, 140.6, 135.0, 134.0, 132.6, 132.4, 132.2, 130.8, 129.5, 128.9, 128.5, 128.4, 128.4, 127.0, 125.4, 124.5, 122.3, 52.8, 19.2

LC-MS (AP2): purity (UV/MS): 98/100, Rₜ 6.93 min

### Example 4

### N-[2-[(2E)-2-(3-bromobenzylidene)hydrazino]-2-oxo-1-(4-oxo-3,4-dihydro-1-phthalazinyl)ethyl]benzamide (4)

N-[2-hydrazino-2-oxo-1-(4-oxo-3,4-dihydro-1-phthalazinyl)ethyl]benzamide (337 mg, 1 mmol) and 3-bromobenzaldehyde (740 mg, 4 mmol) were taken up in ethanol (4 mL) and acetic acid (0.4 mL) and held at 80° C. for 16 h. After cooling to rt, the precipitate was filtered and washed with ethanol, then dried under vacuum. Yield: 431 mg.

LC-MS (AP2): purity (UV/MS): 97/100, Rₜ 7.38 min

### Example 5

### N-[2-[(2E)-2-(2-furylmethylene)hydrazino]-2-oxo-1-(4-oxo-3,4-dihydro-1-phthalazinyl)ethyl]benzamide (5)

N-[2-hydrazino-2-oxo-1-(4-oxo-3,4-dihydro-1-phthalazinyl)ethyl]benzamide (337 mg, 1 mmol) and 2-furaldehyde (384 mg, 4 mmol) were taken up in ethanol (4 mL) and acetic acid (0.4 mL) and held at 80° C. for 16 h. After cooling to rt, the precipitate was filtered and washed with ethanol, then dried under vacuum. Yield: 305 mg.

¹H NMR (400 MHz, DMSO) δ 12.65 (s, 1H), 11.57 (s, 1H), 9.12 (d, J=8.4 Hz, 1H), 8.29 (t, J=8.8 Hz, 1H), 7.99-7.78 (m, 4H), 7.52-7.42 (m, 4H), 6.92 (d, J=8.0 Hz, 1H), 6.41-6.35 (m, 2H)

LC-MS (AP2): purity (UV/MS): 100/74, Rₜ 3.82 min

### Example 6

### N-[2-{(2E)-2-[1-(3-methoxyphenyl)ethylidene]hydrazino}-2-oxo-1-(4-oxo-3,4-dihydro-1-phthalazinyl)ethyl]benzamide (6)

N-[2-hydrazino-2-oxo-1-(4-oxo-3,4-dihydro-1-phthalazinyl)ethyl]benzamide (337 mg, 1 mmol) and 3-methoxyacetophenone (600 mg, 4 mmol) were taken up in ethanol (4 mL) and acetic acid (0.4 mL) and held at 80° C. for 16 h: After cooling to rt, the precipitate was filtered and washed with ethanol, then dried under vacuum. Yield: 419 mg.

¹H NMR (400 MHz, DMSO) δ 12.65 (s, 1H), 10.89 (s, 1H), 9.19 (d, J=8.4 Hz, 1H), 8.27 (d, J=8.0 Hz, 1H), 7.95-8.84 (m, 3H), 7.52 (t, J=7.6 Hz, 1H), 7.43 (t, J=8.0 Hz, 2H), 6.99 (d, J=8.4 Hz, 1H), 6.92 (t, J=8.8 Hz, 1H), 6.80 (d, J=8.0 Hz, 1H), 6.73 (d, J=6.8 Hz, 1H), 3.22 (s, 3H), 2.18 (s, 3H)

¹³C NMR (100 MHz, DMSO), δ 171.1, 167.0, 160.1, 159.7, 147.8, 143.1, 139.8, 134.7, 134.1, 132.4, 132.3, 129.7, 129.6, 128.9, 128.6, 128.4, 126.9, 124.8, 118.9, 115.9, 110.6, 55.1, 52.8, 19.2

LC-MS (AP2): purity (UV/MS): 97/97, Rₜ 6.78 min

### Example 7

### N-[2-[(2E)-2-(2-hydroxybenzylidene)hydrazino]-2-oxo-1-(4-oxo-3,4-dihydro-1-phthalazinyl)ethyl]benzamide (7)

N-[2-hydrazino-2-oxo-1-(4-oxo-3,4-dihydro-1-phthalazinyl)ethyl]benzamide (337 mg, 1 mmol) and salicylaldehyde (488 mg, 4 mmol) were taken up in ethanol (4 mL) and acetic acid (0.4 mL) and held at 80° C. for 16 h. After cooling to rt, the precipitate was filtered and washed with ethanol, then dried under vacuum. Yield: 293 mg.

LC-MS (AP2): purity (UV/MS): 98/88, Rₜ 6.75 min

### Example 8

### N-[2-[(2E)-2-(2-bromobenzylidene)hydrazino]-2-oxo-1-(4-oxo-3,4-dihydro-1-phthalazinyl)ethyl]benzamide (8)

N-[2-hydrazino-2-oxo-1-(4-oxo-3,4-dihydro-1-phthalazinyl)ethyl]benzamide (337 mg, 1 mmol) and 2-bromobenzaldehyde (740 mg, 4 mmol) were taken up in ethanol (4 mL) and acetic acid (0.4 mL) and held at 80° C. for 16 h. After cooling to rt, the precipitate was filtered and washed with ethanol, then dried under vacuum. Yield: 295 mg.

LC-MS (AP2): purity (UV/MS): 100/100, Rₜ 7.99 min

### Example 9

### N-[2-{(2E)-2-[1-(4-bromophenyl)ethylidene]hydrazino}-2-oxo-1-(4-oxo-3,4-dihydro-1-phthalazinyl)ethyl]benzamide (9)

N-[2-hydrazino-2-oxo-1-(4-oxo-3,4-dihydro-1-phthalazinyl)ethyl]benzamide (337 mg, 1 mmol) and 4-bromoacetophenone (796 mg, 4 mmol) were taken up in ethanol (4 mL) and acetic acid (0.4 mL) and held at 80° C. for 16 h. After cooling to rt, the precipitate was filtered and washed with ethanol, then dried under vacuum. Yield: 39 mg.

LC-MS (AP2): purity (UV/MS): 97/89, Rₜ 8.50 min

### Example 10

### N-[2-{(2E)-2-[1-(2-methoxyphenyl)ethylidene]hydrazino}-2-oxo-1-(4-oxo-3,4-dihydro-1-phthalazinyl)ethyl]benzamide (10)

N-[2-hydrazino-2-oxo-1-(4-oxo-3,4-dihydro-1-phthalazinyl)ethyl]benzamide (337 mg, 1 mmol) and 2-methoxyacetophenone (600 mg, 4 mmol) were taken up in ethanol (4 mL) and acetic acid (0.4 mL) and held at 80° C. for 16 h. After cooling to rt, the precipitate was filtered and washed with ethanol, then dried under vacuum. Yield: 133 mg.

LC-MS (AP2): purity (UV/MS): 96/60, Rₜ 6.78 min

In analogous fashion, the following compounds were prepared:

### N-[2-[(2E)-2-(6-methoxy-2,3-dihydro-1H-inden-1-ylidene)hydrazino]-2-oxo-1-(4-oxo-3,4-dihydro-1-phthalazinyl)ethyl]benzamide (11)

LC-MS (AP2): purity (UV/MS): 98/69, Rₜ 7.64 min

### N-[2-[(2E)-2-(4-fluorobenzylidene)hydrazino]-2-oxo-1-(4-oxo-3,4-dihydro-1-phthalazinyl)ethyl]benzamide (12)

LC-MS (AP2): purity (UV/MS): 99/98, Rₜ 7.31 min

### N-[2-{(2E)-2-[1-(2-fluorophenyl)ethylidene]hydrazino}-2-oxo-1-(4-oxo-3,4-dihydro-1-phthalazinyl)ethyl]benzamide (13)

¹H NMR (400 MHz, DMSO) δ 12.69 (s, 1H), 11.01, (s, 1H), 9.16 (d, J=8.4 Hz, 1H), 8.29 (d, J=8.4 Hz, 1H), 7.97-7.83 (m, 4H), 7.52 (t, J=7.2 Hz, 1H), 7.43 (t, J=7.4 Hz, 2H), 7.20-7.16 (m, 1H), 6.99-6.95 (m, 1H), 6.67 (dt, J=7.6 and 2.0 Hz, 1H), 6.60 (t, J=7.6 Hz, 1H), 2.18 (s, 3H)

¹³C NMR (100 MHz, DMSO), δ 172.2, 167.9, 162.5, 161.3 (d, J=240 Hz), 161.0, 160.0, 146.6, 144.3, 135.3, 134.9, 133.2, 132.1 (d, J=8.4 Hz), 130.5, 130.1 (d, J=3.1 Hz), 129.8, 129.7, 129.4, 129.2, 129.1, 128.0 (d, J=10.5 Hz), 127.6, 125.6, 125.1 (d, J=3.4 Hz), 117.5 (d, J=22.1 Hz), 53.4, 18.2

LC-MS (AP2): purity (UV/MS): 99/100, Rₜ 6.83 min

### N-[2-{(2E)-2-[1-(2-bromophenyl)ethylidene]hydrazino}-2-oxo-1-(4-oxo-3,4-dihydro-1-phthalazinyl)ethyl]benzamide (14)

LC-MS (AP2): purity (UV/MS): 99/94, Rₜ 7.18 min

### N-[2-[(2E)-2-(2-fluorobenzylidene)hydrazino]-2-oxo-1-(4-oxo-3,4-dihydro-1-phthalazinyl)ethyl]benzamide (15)

LC-MS (AP2): purity (UV/MS): 100/100, Rₜ 7.22 min

### N-(2-oxo-1-(4-oxo-3,4-dihydro-1-phthalazinyl)-2-{(2E)-2-[3-(trifluoromethyl)-benzylidene]hydrazino}ethyl)benzamide (16)

LC-MS (AP2): purity (UV/MS): 100/75, Rₜ 8.33 min

### N-{2-oxo-1-(4-oxo-3,4-dihydro-1-phthalazinyl)-2-[(2E)-2-(2-pyridinylmethylene)-hydrazino]ethyl}benzamide (17)

LC-MS (AP2): purity (UV/MS): 93/96, Rₜ 2.93 min

### N-{2-oxo-1-(4-oxo-3,4-dihydro-1-phthalazinyl)-2-[(2E)-2-(3-pyridinylmethylene)-hydrazino]ethyl}benzamide (18)

¹H NMR (400 MHz, DMSO) δ 12.66 (s, 1H), 11.79 (s, 1H), 9.17 (d, J=8.4 Hz, 1H), 8.39 (dd, J=4.8 and 1.6 Hz, 1H), 8.31 (d, J=1.6 Hz, 1H), 8.28 (d, J=8.0, 1H), 8.04-7.91 (m, 5H), 7.54 (dt, J=7.2 and 1.2 Hz, 1H), 7.47-7.43 (m, 2H), 7.36 (td, J=8.0 and 1.6 Hz, 1H), 7.11 (dd, J=8.0 and 4.8 Hz, 1H), 6.95 (d, J=8.4 Hz, 1H)

LC-MS (AP2): purity (UV/MS): 100/98, Rₜ 2.22 min

### N-[2-((2E)-2-{[3-chloro-5-(trifluoromethyl)-2-pyridinyl]methylene}-hydrazino)-2-oxo-1-(4-oxo-3,4-dihydro-1-phthalazinyl)ethyl]benzamide (19)

¹H NMR (400 MHz, DMSO) δ 12.69 (s, 1H), 9.28 (d, J=8.4 Hz, 1H), 8.76 (d, J=1.2 Hz, 1H), 8.28-8.25 (m, 2H), 8.21 (s, 1H), 7.95-7.91 (m, 2H), 7.86 (t, J=7.6 Hz, 2H), 7.56-7.51 (m, 1H), 7.45 (t, J=7.6 Hz, 2H), 6.95 (d, J=8.4 Hz, 1H)

LC-MS (AP2): purity (UV/MS): 100/100, Rₜ 7.25 min

### N-(2-oxo-1-(4-oxo-3,4-dihydro-1-phthalazinyl)-2-{(2E)-2-[1-(3-pyridinyl)-ethylidene]hydrazino}ethyl)benzamide (20)

LC-MS (AP2): purity (UV/MS): 100/100, Rₜ 2.42 min

### N-(2-oxo-1-(4-oxo-3,4-dihydro-1-phthalazinyl)-2-{(2E)-2-[1-(2-pyridinyl)ethylidene]hydrazino}ethyl)benzamide (21)

LC-MS (AP2): purity (UV/MS): 99/100, Rₜ 3.97 min

### N-[2-[(2E)-2-(1,3-dimethylbutylidene)hydrazino]-2-oxo-1-(4-oxo-3,4-dihydro-1-phthalazinyl)ethyl]benzamide (22)

LC-MS (AP2): purity (UV/MS): 96/83, Rₜ 6.66 min

### Methyl (4E)-4-{[(benzoylamino)(4-oxo-3,4-dihydro-1-phthalazinyl)acetyl]-hydrazono}pentanoate (23)

LC-MS (AP2): purity (UV/MS): 98/78, Rₜ 4.62 min

### N-[2-{(2E)-2-[2-(3-methoxyphenyl)-1-methylethylidene]hydrazino}-2-oxo-1-(4-oxo-3,4-dihydro-1-phthalazinyl)ethyl]benzamide (24)

LC-MS (AP2): purity (UV/MS): 98/99, Rₜ 6.85 min

### N-[2-{(2E)-2-[1-(3-bromophenyl)propylidene]hydrazino}-2-oxo-1-(4-oxo-3,4-dihydro-1-phthalazinyl)ethyl]benzamide (25)

¹H NMR (400 MHz, DMSO) δ 12.65 (s, 1H), 11.11 (s, 1H), 9.20 (d, J=8.8 Hz, 1H), 8.27 (dd, J=8.0 and 0.8 Hz, 1H), 7.99-7.92 (m, 3H), 7.55-7.51 (m, 1H), 7.46-7.42 (m, 2H), 7.35-7.32 (m, 1H), 7.27-7.24 (m, 1H), 7.22 (t, J=2.0 Hz, 1H), 7.02-6.98 (m, 2H), 2.72 (q, J=7.6 Hz, 2H), 0.95 (t, J=7.6 Hz, 3H)

¹³C NMR (100 MHz, DMSO), 6171.3, 167.1, 160.1, 150.9, 143.2, 139.4, 135.0, 134.0, 132.6, 132.4, 132.2, 130.9, 129.5, 128.9, 128.5, 128.4, 128.3, 127.0, 125.4, 124.5, 122.4, 52.8, 19.6, 11.4

LC-MS (AP2): purity (UV/MS): 99/100, Rₜ 7.94 min

### N-[2-{(2E)-2-[1-(3-fluorophenyl)ethylidene]hydrazino}-2-oxo-1-(4-oxo-3,4-dihydro-1-phthalazinyl)ethyl]benzamide (26)

LC-MS (AP2): purity (UV/MS): 100/100, Rₜ 6.96 min

### N-[2-oxo-1-(4-oxo-3,4-dihydro-1-phthalazinyl)-2-((2E)-2-{1-[3-(trifluoromethyl)phenyl]ethylidene}hydrazino)ethyl]benzamide (27)

LC-MS (AP2): purity (UV/MS): 96/87, Rₜ 7.80 min

### N-[2-{(2E)-2-[1-(3-chlorophenyl)ethylidene]hydrazino}-2-oxo-1-(4-oxo-3,4-dihydro-1-phthalazinyl)ethyl]benzamide (28)

¹H NMR (400 MHz, DMSO) δ 12.67 (s, 1H), 9.21 (d, J=8.8 Hz, 1H), 8.30 (d, J=8.0 Hz, 1H), 8.00-7.87 (m, 4H), 7.53 (t, J=7.2 Hz, 1H), 7.44 (t, J=8.0 Hz, 2H), 7.20-7.17 (m, 2H), 7.07-7.00 (m, 3H), 2.19 (s, 3H)

¹³C NMR (100 MHz, DMSO), δ 171.3, 167.1, 160.2, 146.4, 143.3, 140.4, 135.0, 134.0, 133.8, 132.6, 132.4, 130.5, 129.6, 129.3, 128.9, 128.6, 128.4, 127.0, 125.5, 125.0, 124.5, 52.8, 19.2

LC-MS (AP2): purity (UV/MS): 100/100, Rₜ 7.45 min

### N-[2-{(2E)-2-[1-(3-cyanophenyl)ethylidene]hydrazino}-2-oxo-1-(4-oxo-3,4-dihydro-1-phthalazinyl)ethyl]benzamide (29)

LC-MS (AP2): purity (UV/MS): 90/100, Rₜ 6.20 min

### N-[2-{(2E)-2-[1-(3-hydroxyphenyl)ethylidene]hydrazino}-2-oxo-1-(4-oxo-3,4-dihydro-1-phthalazinyl)ethyl]benzamide (30)

LC-MS (AP2): purity (UV/MS): 99/100, Rₜ 3.97 min

### N-[2-{(2E)-2-[1-(3-methyl phenyl)ethylidene]hydrazino}-2-oxo-1-(4-oxo-3,4-dihydro-1-phthalazinyl)ethyl]benzamide (31)

¹H NMR (400 MHz, DMSO) δ 12.66 (s, 1H), 10.89 (s, 1H), 9.19 (d, J=8.8 Hz, 1H), 8.30 (dd, J=8.0 and 0.8 Hz, 1H), 8.00-7.81 (m, 4H), 7.55-7.51 (m, 1H), 7.44 (t, J=8.0 Hz, 2H), 7.06-6.92 (m, 3H), 6.84 (s, 1H)

¹³C NMR (100 MHz, DMSO), δ 171.2, 167.1, 160.1, 147.8, 143.4, 138.3, 137.7, 134.8, 134.0, 132.5, 132.4, 130.2, 129.7, 128.9, 128.6, 128.5, 128.4, 126.9, 126.3, 124.7, 123.7, 52.8, 21.2, 14.0

LC-MS (AP2): purity (UV/MS): 100/100, Rₜ 7.27 min

### N-[2-{(2E)-2-[1-(4-methyl-2-pyridinyl)ethylidene]hydrazino}-2-oxo-1-(4-oxo-3,4-dihydro-1-phthalazinyl)ethyl]benzamide (32)

¹H NMR (400 MHz, DMSO) δ 12.67 (s, 1H), 11.06 (s, 1H), 9.22 (d, J=8.4 Hz, 1H), 8.31-7.91 (m, 5H), 7.53 (t, J=7.6 Hz, 1H), 7.44 (t, J=7.6 Hz, 2H), 7.00 (t, J=8.4 Hz, 2H), 6.68 (s, 1H), 2.25 (s, 3H), 1.60 (s, 3H)

LC-MS (AP2): purity (UV/MS): 100/96, Rₜ 5.57 min

### N-[2-{(2E)-2-[1-(3-bromophenyl)-2,2-dimethylpropylidene]-hydrazino}-2-oxo-1-(4-oxo-3,4-dihydro-1-phthalazinyl)ethyl]benzamide (33)

¹H NMR (400 MHz, CD₃OD) δ 8.80 (dd, J=7.6 and 1.2 Hz, 1H), 8.15 (d, J=8.0 Hz, 1H), 7.96 (dt, J=8.0 and 1.2 Hz, 1H), 7.90-7.85 (m, 4H), 7.63-7.61 (m, 1H), 7.50 (dt, J=7.6 and 1.2 Hz, 1H), 7.43-7.39 (m, 4H), 7.14 (s, 1H), 0.74 (s, 9H)

LC-MS (AP2): purity (UV/MS): 99/97, Rₜ 8.74 min

### N-[2-{(2E)-2-[1-(3-bromophenyl)-2-methylpropylidene]hydrazino}-2-oxo-1-(4-oxo-3,4-dihydro-1-phthalazinyl)ethyl]benzamide (34)

LC-MS (AP2): purity (UV/MS): 96/75, Rₜ 8.31 min

### N-[2-{(2E)-2-[(3-bromophenyl)(cyclopropyl)methylene]hydrazino}-2-oxo-1-(4-oxo-3,4-dihydro-1-phthalazinyl)ethyl]benzamide (35)

LC-MS (AP2): purity (UV/MS): 97/91, Rₜ 8.12 min

### N-[2-{(2E)-2-[1-(3-bromophenyl)butylidene]hydrazino}-2-oxo-1-(4-oxo-3,4-dihydro-1-phthalazinyl)ethyl]benzamide (36)

LC-MS (AP2): purity (UV/MS): 98/90, Rₜ 8.40 min

### N-[2-[(2E)-2-(2,2-dimethyl-1-phenylpropylidene)hydrazino]-2-oxo-(4-oxo-3,4-dihydro-1-phthalazinyl)ethyl]benzamide (37)

¹H NMR (400 MHz, DMSO) δ 8.39 (d, J=8.0 Hz, 1H), 8.16 (d, J=8.4 Hz, 1H), 7.96 (dt, J=8.0 and 1.6 Hz, 1H), 7.91-7.87 (m, 4H), 7.52-7.40 (m, 7H), 7.16 (s, 1H), 0.74 (s, 9H)

LC-MS (AP2): purity (UV/MS): 99/76, Rₜ 8.11 min

### N-[2-{(2E)-2-[1-(3,5-dimethoxyphenyl)ethylidene]hydrazino}-2-oxo-1-(4-oxo-3,4-dihydro-1-phthalazinyl)ethyl]benzamide (38)

LC-MS (AP2): purity (UV/MS): 99/100, Rₜ 6.96 min

### N-[2-((2E)-2-{1-[3,5-bis(trifluoromethyl)phenyl]ethylidene}-hydrazino)-2-oxo-1-(4-oxo-3,4-dihydro-1-phthalazinyl)ethyl]benzamide (39)

¹H NMR (400 MHz, DMSO) δ 12.64 (s, 1H), 11.2 (s, 1H), 9.24 (d, J=8.4 Hz, 1H), 8.26 (d, J=7.6 Hz, 1H), 7.94-7.82 (m, 6H), 7.53 (t, J=7.2 Hz, 1H), 7.44 (t, J=7.6 Hz, 2H), 6.99 (d, J=8.4 Hz, 1H), 2.32 (s, 3H)

LC-MS (AP2): purity (UV/MS): 100/100, Rₜ 8.56 min

### N-[2-{(2E)-2-[1-(3,5-difluorophenyl)ethylidene]hydrazino}-2-oxo-1-(4-oxo-3,4-dihydro-1-phthalazinyl)ethyl]benzamide (40)

¹H NMR (400 MHz, DMSO) δ 12.65 (s, 1H), 11.07 (s, 1H), 9.21 (d, J=8.8 Hz, 1H), 8.27 (d, J=8.0 Hz, 1H), 7.96-7.87 (m, 4H), 7.61 (d, J=6.0 Hz, 1H), 7.53 (t, J=7.2 Hz, 1H), 7.44 (t, J=7.6 Hz, 2H), 6.97 (t, J=7.6 Hz, 1H), 6.77 (d, J=6.8 Hz, 1H), 2.17 (s, 3H)

LC-MS (AP2): purity (UV/MS): 90/100, Rₜ 7.32 min

### N-[2-[(2E)-2-(3,5-dibromobenzylidene)hydrazino]-2-oxo-1-(4-oxo-3,4-dihydro-1-phthalazinyl)ethyl]benzamide (41)

¹H NMR (400 MHz, DMSO) δ 12.65 (bs, 1H), 9.17 (d, J=8.0 Hz, 1H), 8.28 (d, J=7.6 Hz, 1H), 8.07-7.89 (m, 4H), 7.79 (s, 1H), 7.63 (d, J=1.2 Hz, 1H), 7.53 (t, J=6.8 Hz, 1H), 7.44 (t, J=7.6 Hz, 2H), 7.24 (s, 2H), 6.91 (d, J=8.4 Hz, 1H)

LC-MS (AP2): purity (UV/MS): 99/86, Rₜ 8.23 min

### N-[2-{(2E)-2-[1-(3,5-dibromophenyl)ethylidene]hydrazino}-2-oxo-1-(4-oxo-3,4-dihydro-1-phthalazinyl)ethyl]benzamide (42)

¹H NMR (400 MHz, DMSO) δ 12.66 (s, 1H), 10.89 (s, 1H), 9.21 (d, J=7.6 Hz, 1H), 8.26 (d, J=7.6 Hz, 1H), 7.94-7.90 (m, 2H), 7.87 (t, J=8.0 Hz, 1H), 7.52 (t, J=7.2 Hz, 1H), 7.43 (t, J=7.6 Hz, 2H), 6.97 (d, J=8.4 Hz, 1H), 6.41 (d, J=2.4 Hz, 2H), 6.30 (t, J=2.0 Hz, 1H), 2.21 (s, 3H)

LC-MS (AP2): purity (UV/MS): 100/86, Rₜ 8.40 min

### N-[2-{(2E)-2-[1-(3,5-dimethylphenyl)ethylidene]hydrazino}-2-oxo-1-(4-oxo-3,4-dihydro-1-phthalazinyl)ethyl]benzamide (43)

¹H NMR (400 MHz, DMSO) δ 12.65 (s, 1H), 10.86 (s, 1H), 9.18 (d, J=8.8 Hz, 1H), 8.30 (d, J=8.0 Hz, 1H), 8.00-7.88 (m, 4H), 7.53 (t, J=7.2 Hz, 1H), 7.44 (t, J=7.6 Hz, 2H), 6.97 (d, J=8.8 Hz, 1H), 6.75 (s, 2H), 2.15 (s, 3H), 1.84 (s, 6H)

LC-MS (AP2): purity (UV/MS): 100/96, Rₜ 7.77 min

### Example 11

### Receptor Selection and Amplification Technology Assay

The functional receptor assay, Receptor Selection and Amplification Technology (R-SAT), was used to investigate the pharmacological properties of known and novel PAR2 compounds. R-SAT is disclosed in U.S. Pat. Nos. 5,707,798, 5,912,132, and 5,955,281, all of which are hereby incorporated herein by reference in their entirety, including any drawings.

Briefly, NIH3T3 cells were grown in 96 well tissue culture plates to 70-80% confluence. Cells were transfected for 16-20 h with plasmid DNAs using Polyfect (Qiagen Inc.) as per manufacturer's protocols. R-SATs were generally performed with 4 ng/well of receptor and 20 ng/well of β-galactosidase plasmid DNA. All receptor constructs used were in the pSI-derived mammalian expression vector (Promega Inc) as described previously. The PAR2 gene was amplified by PCR from genomic DNA using oligodeoxynucleotide primers based on the published sequence (GenBank Accession # Z49993 and Z49994). For large-scale transfections, cells were transfected for 16-20 h, then trypsinized and frozen in DMSO. Frozen cells were later thawed, plated at ˜10,000 cells per well of a 96 half-area well plate that contained drug. With both methods, cells were then grown in a humidified atmosphere with 5% ambient CO₂ for five days. Media was then removed from the plates and marker gene activity was measured by the addition of the β-galactosidase substrate o-nitrophenyl β-D-galactopyranoside (ONPG, in PBS with 0.5% NP-40). The resulting colorimetric reaction was measured in a spectrophotometric plate reader (Titertek Inc.) at 420 nm. All data were analyzed using the computer program XLFit (IDBSm). Efficacy is the percent maximal activation compared to activation by a control compound, the PAR2 activating hexapeptide SLIGRL (serine-leucine-isoleucine-glycine-arginine-leucine). pEC₅₀ is the negative of the log (EC₅₀), where EC₅₀ is the calculated concentration in Molar that produces 50% maximal activation.

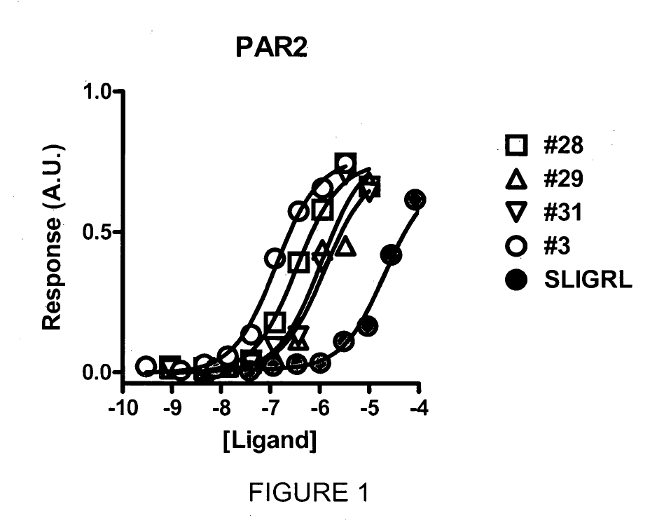

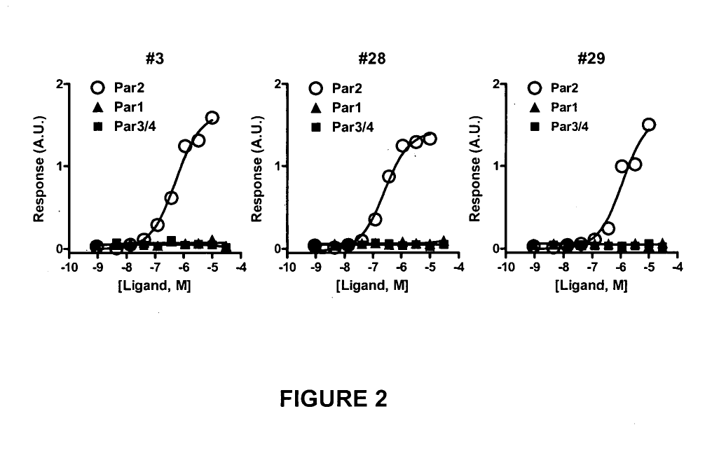

These experiments provide a molecular profile, or fingerprint, for each of these agents at human PAR2. As can be seen in Table 1, FIG. 1, and FIG. 2, these compounds activate PAR2.

TABLE 1

| Compound | pEC₅₀ | % Efficacy |
|---|---|---|
| 3 | 6.9 | 98 |
| 43 | 7.1 | 122 |
| 28 | 6.8 | 112 |
| 27 | 6.6 | 77 |
| 31 | 6.5 | 118 |
| 29 | 6.5 | 132 |
| 25 | 6.3 | 111 |
| 6 | 6.1 | 79 |

Efficacy is relative to that of SLIGRL.

### Example 12

### PAR2 Binding Assay

Using the following materials and methods, the ability of the compounds disclosed herein to bind to PAR2 can be readily determined in a receptor binding assay.

1. Grow PAR2-transfected COS cells (or another transfected cell line that does not endogenously express PAR2 may be substituted) in a suitable growth medium in 24-well culture plates.

2. Prepare radiolabeled assay solutions by mixing 245 μl of 0.25 nM [¹²⁵I] SLIGRL working solution with 5 μl of the following (one per solution): 50 μM unlabeled SLIGRL working solution, 0.25 nM [¹²⁵I] SLIGRL working solution, HEPES buffer only, or 50× test compound.

3. Aspirate medium from 24-well plates using a Pasteur pipet attached to a vacuum source. Do not wash cells.

4. Add 250 μl radiolabeled assay solution from step 2 to each assay well and incubate plates 60 min at room temperature (˜22° C.) on an orbital shaker at low speed.

5. Terminate the incubation by aspirating the radioactive solution with a 24-well Brandel cell harvester. Wash the wells three times with 0.5 ml ice-cold HEPES buffer using the cell harvester.

6. Aspirate the solution from the wells with a micropipettor and transfer to 12×75-mm polystyrene test tubes. Analyze with a gamma counter (Packard, Cobra II).

7. Determine specific binding and calculate the IC₅₀ values.

### Example 13

### Determination of Changes in Cytosolic Calcium in Transfected CHO-K1 Cells

1. CHO-K1 cells transfected with PAR2 or a control receptor at a density 1-3×10⁶ cells/ml are washed with phosphate-buffered saline.

2. Cells are loaded with 2 μM Fura-2 and analyzed with respect to the rise in intracellular calcium in the presence or absence of varying concentration of compound.

3. The response is compared to that elicited by the application of the standard reference ligand SLIGRL when tested at 100 nM.

Intracellular free calcium concentrations are calculated using the formula:

$${[ {Ca}^{2 +} ]}_{i} = \frac{{K}_{d} ( F - {F}_{min} )}{{F}_{max} - F}$$

where K_{d} for Fura-2 is 224 nM, Fₘₐₓ is the fluorescence in the presence of 0.04% Triton-X100 and Fₘᵢₙ is the fluorescence obtained after the addition of 5 mM EGTA in 30 mM Tris-HCl, pH7.4.

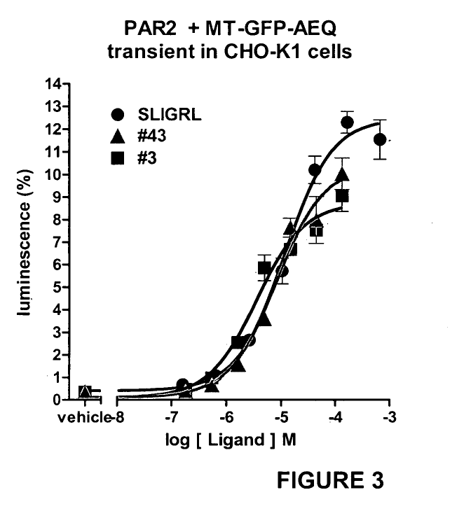

As shown in Table 2 and FIG. 3, these experiments have shown these agents act at human PAR2 to stimulate intracellular calcium mobilization.

TABLE 2

| Compound | pEC₅₀ | % Efficacy |
|---|---|---|
| 3 | 5.4 | 72 |
| 43 | 5.1 | 85 |

### Example 14

### Determination of Changes in Inositol Phosphates in Transfected TsA Cells

Seed tsA cells (a transformed HEK293 cell line) at 10,000 cells/0.1 ml per well of 96 well plates at 37 C in a humidified 5% CO2 incubator in DMEM supplemented with 10% fetal calf serum, penicillin (100 units/ml) and streptomycin (100 mg/ml) and grown overnight.

Transfect the cells with plasmid DNAs coding receptors or G-protein helpers when needed using PolyFect according to the RSAT protocol described previously. At 18-20 h post-transfection, the medium is removed and the cells are labeled overnight with 2 uCi/ml myo-[2-3H] inositol (0.1 ml/well) freshly made in the culture medium.

Remove the medium and wash cells with Hank's Balanced Salt Solutions (HBSS) containing 1 mM CaCl2, 1 mM MgCl2, 20 mM LiCl and 0.1% BSA. The cells are then incubated with ligands for 45 min at 37 C (0.1 ml/well) and the reaction is stopped by exchanging the buffer with 150 ul/well ice-cold 20 mM formic acid. Add 50 ul/well 0.2M ammonium and store plates at −80 C or process samples immediately.

To separate total [3H] inositol phosphates (IPs) ion-exchange chromatography columns are loaded with 200 ul of AG 1-X8 resin suspension (50% resin and 50% water) and the cell extracts were applied to the columns. Wash the columns with 1 ml 40 mM ammonium hydroxide (pH9) and elute [3H] IPs into the 2 ml deep-well blocks with 0.4 ml 2M ammonium format/0.1M formic acid. Wash the columns with 0.6 ml water. Transfer the eluates into 7 ml scintillation vials and add 5 ml liquid scintillation cocktail. Mix well, leave the vials in the dark for at least 4 h and count on LS 6500 Multi-purpose Scintillation Counter (3 min/vial). This procedure collects IP1, IP2 and IP3.

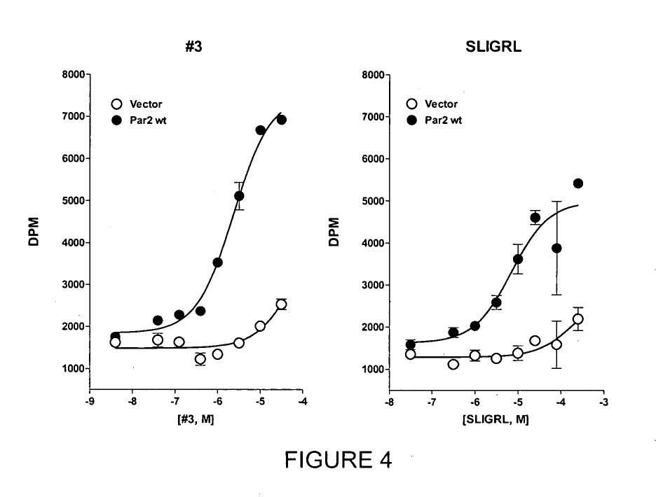

As shown in Table 3 and FIG. 4, these experiments have shown these agents act at human PAR2 to stimulate inositol phosphate hydrolysis.

TABLE 3

| Compound | pEC₅₀ | % Efficacy |
|---|---|---|
| 3 | 5.6 | 120 |
| 43 | 5.0 | 130 |

### Example 15

### Sequences for PAR2

The DNA sequence encoding PAR2 (SEQ ID NO:1) and the polypeptide sequence of PAR2 (SEQ ID NO:2) are:

SEQ ID NO:1

ctcgagcggccgccagtgtgatggatatctgcagaattcgccctttgcgt
ccagtggagctctgagtttcgaatcggtggcggcggattccccgcgcgcc
cggcgtcggggcttccaggaggatgcggagccccagcgcagcgtggctgc
tgggggccgccatcctgctagcagcctctctctcctgcagtggcaccatc
caaggaaccaatagatcctctaaaggaagaagccttattggtaaggttga
tggcacatcccacgtcactggaaaaggagttacagttgaaacagtctttt
ctgtggatgagttttctgcatctgtcctcactggaaaactgaccactgtc
ttccttccaattgtctacacaattgtgtttgtggtgggtttgccaagtaa
cggcatggccctgtgggtctttcttttccgaactaagaagaagcaccctg
ctgtgatttacatggccaatctggccttggctgacctcctctctgtcatc
tggttccccttgaagattgcctatcacatacatggcaacaactggattta
tggggaagctctttgtaatgtgcttattggctttttctatggcaacatgt
actgttccattctcttcatgacctgcctcagtgtgcagaggtattgggtc
atcgtgaaccccatggggcactccaggaagaaggcaaacattgccattgg
catctccctggcaatatggctgctgattctgctggtcaccatccctttgt
atgtcgtgaagcagaccatcttcattcctgccctgaacatcacgacctgt
catgatgttttgcctgagcagctcttggtgggagacatgttcaattactt
cctctctctggccattggggtctttctgttcccagccttcctcacagcct
ctgcctatgtgctgatgatcagaatgctgcgatcttctgccatggatgaa
aactcagagaagaaaaggaagagggccatcaaactcattgtcactgtcct
ggccatgtacctgatctgcttcactcctagtaaccttctgcttgtggtgc
attattttctgattaagagccagggccagagccatgtctatgccctgtac
attgtagccctctgcctctctacccttaacagctgcatcgacccctttgt
ctattactttgtttcacatgatttcagggatcatgcaaagaacgctctcc
tttgccgaagtgtccgcactgtaaagcagatgcaagtatccctcacctca
aagaaacactccaggaaatccagctcttactcttcaagttcaaccactgt
taagacctcctattgagttttccaggtcctcagatgggaattgcacagta
ggatgtggaacctgtttaatgttatgaggacgtgtctgaagggcgaattc
cagcacactggcggccgtt

SEQ ID NO:2

MRSPSAAWLLGAAILLAASLSCSGTIQGTNRSSKGRSLIGKVDGTSHVTG
KGVTVETVFSVDEFSASVLTGKLTTVFLPIVYTIVFVVGLPSNGMALWVF
LFRTKKKHPAVIYMANLALADLLSVIWFPLKIAYHIHGNNWIYGEALCNV
LIGFFYGNMYCSILFMTCLSVQRYWVIVNPMGHSRKKANIAIGISLAIWL
LILLVTIPLYVVKQTIFIPALNITTCHDVLPEQLLVGDMFNYFLSLAIGV
FLFPAFLTASAYVLMIRMLRSSAMDENSEKKRKRAIKLIVTVLAMYLICF
TPSNLLLVVHYFLIKSQGQSHVYALYIVALCLSTLNSCIDPFVYYFVSHD
FRDHAKNALLCRSVRTVKQMQVSLTSKKHSRKSSSYSSSSTTVKTSY*

What is claimed:

1. A compound having the chemical structure: 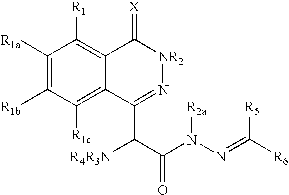
or a pharmaceutically acceptable salt or prodrug thereof, wherein:
R₁, R₁ₐ, R_{1b} and R_{1c} are independently selected from the group consisting of hydrogen, C₁-C₁₀ alkyl, C₃-C₆ cycloalkyl, substituted or unsubstituted aryl, substituted or unsubstituted heteroaryl, hydroxy, nitro, amino, halogen, sulfonate, perhaloalkyl, —OR₇, —NR₇R₇ₐ, —CN, —C(=Z)R₇, —C(=Z)OR₇, —C(=Z)NR₇R₇ₐ, —N(R₇)—C(=Z)R₇ₐ, —N(R₇)—C(=Z)NR₇ₐR_{7b}, —OC(=Z)R₇, and —SR₇, wherein:
Z is oxygen or sulfur;
R₇, R₇ₐ and R_{7b} are independently selected from the group consisting of:
hydrogen;
unsubstituted or substituted C₁-C₁₀ alkyl;
C₁-C₁₀ alkyl substituted with a group selected from the group consisting of aryl and heteroaryl; unsubstituted C₂-C₁₀ alkenyl;
C₂-C₁₀ alkenyl substituted with a group selected from the group consisting of aryl or heteroaryl;
unsubstituted C₂-C₁₀ alkynyl;
C₂-C₁₀ alkynyl substituted with a group selected from the group consisting of aryl or heteroaryl,
C₃-C₇ cycloalkyl, and
C₅-C₇ cycloalkenyl; or,
R₁ and R₁ₐ, R₁ₐ and R_{1b} or R_{1b} and R_{1c}, taken together, form a fused aryl or heteroaryl ring;
X is selected from the group consisting of oxygen, sulfur, nitrogen and NR₈, wherein:
R₈ is selected from the group consisting of C₁-C₄ alkyl, C₃-C₇ cycloalkyl, substituted or unsubstituted aryl, substituted and unsubstituted heteroaryl;
R₂ and R₂ₐ are independently selected from the group consisting of hydrogen, unsubstituted or substituted C₁-C₄ alkyl, unsubstituted or substituted C₃-C₇ cycloalkyl, unsubstituted or substituted aryl and unsubstituted or substituted heteroaryl;
R₃ and R₄ are independently selected from the group consisting of hydrogen, unsubstituted or substituted C₁-C₄ alkyl, unsubstituted or substituted C₃-C₇ cycloalkyl, unsubstituted or substituted aryl and unsubstituted or substituted heteroaryl or COR₁₀; wherein:
R₁₀ is independently selected from the group consisting of C₁-C₅ alkyl, unsubstituted or substituted aryl and unsubstituted or substituted heteroaryl; or,
R₃ and R₄, taken together, form an unsubstituted or substituted 5-, 6-, 7- or 8-member ring;
R₅ and R₆ are independently selected from the group consisting of hydrogen, unsubstituted or substituted C₁-C₁₀ alkyl and substituted or unsubstituted C₃-C₇ cycloalkyl, unsubstituted or substituted aryl and substituted or unsubstituted heteroaryl; or,
R₅ and R₆, taken together, form a 5-, 6-, 7- or 8-member cycloalkyl fused to a group selected from the group consisting of unsubstituted or substituted aryl and unsubstituted or substituted heteroaryl.

2. The compound of claim 1, wherein R₅ is selected from the group consisting of hydrogen, methyl and cyclopropyl.

3. The compound of claim 1, wherein R₅ is substituted with 1-4 groups selected from the group consisting of hydroxy, halogen, perhaloalkyl, —OR₁₁, —NR₁₁ R₁₁ₐ, —CN, —C(X)R₁₁, —C(X)OR₁₁, —C(X)NR₁₁R₁₁ₐ, —N(R₁₁)—C(X)R₁₁ₐ, —N(R₁₁)—C(X)NR₁₁R₁₁ₐ and —OC(X)R₁₁, wherein:
X is oxygen; and,
R₁₁ and R₁₁ₐ are independently selected from the group consisting of hydrogen, C₁-C₄ alkyl, C₂-C₄ alkenyl, C₂-C₄ alkynyl, C₃-C₇ cycloalkyl, and C₅-C₁₀ cycloalkenyl.

4. The compound of claim 1, wherein R₆ is unsubstituted or substituted phenyl wherein, if substituted, the substituent is one or more selected from the group consisting of C₁-C₄ alkyl, hydroxyl, halogen, perhaloalkyl, and OR₉, wherein:
R₉ is selected from the group consisting of hydrogen, methyl, fluoro, chloro, bromo, —CN, trifluoromethyl.

5. The compound of claim 1, wherein R₆ is unsubstituted or substituted heteroaryl wherein, if substituted, the substituent is one or more independently selected from the group consisting of halogen, C₁-C₁₀alkyl and perhaloalkyl. In an aspect of this invention the heteroaryl is pyridine or furan.

6. The compound of claim 1, wherein the substituent on R₅ is selected from the group consisting of methyl, trifluoromethyl and chloro.

7. The compound of claim 1, wherein R₆ is unsubstituted or substituted C₁-C₁₀ alkyl, wherein, if substituted, the substituent is one more independently selected from the group consisting of unsubstituted C₁-C₅ alkyl and —C(O)OR₁₀, wherein R₁₀ is unsubstituted C₁-C₅ alkyl.

8. The compound of claim 1, wherein R₆ is substituted with 1-4 groups selected from the group consisting of hydroxy, halogen, perhaloalkyl, —OR₁₁, —NR₁₁ R₁₁ₐ, —CN, —C(X)R₁₁, —C(X)OR₁₁, —C(X)NR₁₁R₁₁ₐ, N(R₁₁)—C(X)R₁₁ₐ, —N(R₁₁)—C(X)NR₁₁R₁₁ₐ and —OC(X)R₁₁, wherein:
X is oxygen; and,
R₁₁ and R₁₁ₐ are independently selected from the group consisting of hydrogen, C₁-C₄ alkyl, C₂-C₄ alkenyl, C₂-C₄ alkynyl, C₃-C₇ cycloalkyl, and C₅-C₁₀ cycloalkenyl.

9. The compound of claim 1, wherein R₆ is selected from the group consisting of hydrogen, methyl and cyclopropyl.

10. The compound of claim 1, wherein R₆ is unsubstituted or substituted phenyl wherein, if substituted, the substituent is one or more selected from the group consisting of C₁-C₄ alkyl, hydroxyl, halogen, perhaloalkyl, and OR₉, wherein:
R₉ is selected from the group consisting of hydrogen, methyl, fluoro, chloro, bromo, —CN, trifluoromethyl.

11. The compound of claim 1, wherein R₆ is unsubstituted or substituted heteroaryl wherein, if substituted, the substituent is one or more independently selected from the group consisting of halogen, C₁-C₁₀alkyl and perhaloalkyl.

12. The compound of claim 11, wherein the heteroaryl is pyridine or furan.

13. The compound of claim 11, wherein the substituent on R₆ is selected from the group consisting of methyl, trifluoromethyl and chloro.

14. The compound of claim 1, wherein R₆ is unsubstituted or substituted C₁-C₁₀ alkyl, wherein, if substituted, the substituent is one more independently selected from the group consisting of unsubstituted C₁-C₅alkyl and —C(O)OR₁₀, wherein R₁₀ is unsubstituted C₁-C₅ alkyl.

15. The compound of claim 1 wherein R₁ is hydrogen.

16. The compound of claim 1, wherein R₁ₐ is hydrogen.

17. The compound of claim 1, wherein R_{1b} is hydrogen.

18. The compound of claim 1, wherein, R_{1c} is hydrogen.

19. The compound of claim 1, wherein R₂ is hydrogen.

20. The compound of claim 1, wherein R₂ₐ is hydrogen.

21. The compound of claim 1, wherein R₃ is benzoyl.

22. The compound of claim 1, wherein R₄ is hydrogen.

23. The compound of claim 1, wherein X is oxygen.

24. The compound of claim 1, wherein R₁, R₁ₐ, R_{1b}, R_{1c}, R₂, R₂ₐ and R₄ are hydrogen; R₃ is benzoyl; and, X is oxygen.

25. The compound of claim 1, wherein R₅ and R₆, taken together, form a 2,3-dihydro-1H-indene ring.

26. A method of screening for a compound that modulates the activity of PAR2 or a PAR2 subtype, comprising:
providing a recombinant cell comprising a nucleic acid that expresses PAR2 or a PAR2 subtype;
contacting the recombinant cell with a test compound; and,
detecting changes in the activity of the PAR2 or the PAR2 subtype in the cell.

27. The method of claim 26, further comprising comparing the effect of the compound on the recombinant cell with its effect on a cell that does not contain a nucleic acid that expresses PAR2 or PAR2 subtype.

28. The method of claim 26, wherein the nucleic acid expressing PAR2 or PAR2 subtype comprises at least 20 contiguous nucleotides which hybridizes under stringent hybridization conditions to at least 20 contiguous nucleotides of a complement of nucleic acid SEQ ID NO:1 or of a nucleic acid that encodes a polypeptide having SEQ ID NO:2.

29. The method of claim 26, wherein the nucleic acid expressing PAR2 or PAR2 subtype comprises at least 20 contiguous nucleotides which can hybridize under stringent hybridizations conditions to a complement of at least 20 contiguous nucleotides of a nucleic acid that encodes SEQ ID NO:2.

30. The method of claim 26, wherein the nucleic acid expressing PAR2 or PAR2 subtype comprises at least 50, at least 100, at least 150, at least 200, at least 250, at least 300, at least 350, at least 400, at least 450, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, or at least 2500, contiguous nucleotides which can hybridize under stringent hybridizations conditions to a complement of the same number of contiguous nucleotides of a nucleic acid that encodes the amino acid sequence of SEQ ID NO:2.

31. The method of claim 26, wherein the nucleic acid encodes the polypeptide sequence of SEQ ID NO:2

32. A method of treating or preventing a disease or disorder related to PAR2 activity comprising administering a therapeutically effective amount of a compound of claim 1 to a patient in need thereof.

33. The method of claim 32, wherein the disease or disorder is selected from the group consisting of:
acute or chronic pain;
acute or chronic inflammation;
diseases or disorder of the pulmonary system;
diseases or disorders of the gastrointestinal system;
diseases or disorders of the musculoskeletal system;
diseases or disorders of the central nervous system;
diseases or disorders of the cardiovascular system;
disease or disorders of the renal system;
diseases or disorders of the hepatic system;
diseases of disorders of the eye;
diseases or disorders of the skin;
diseases or disorders of the prostrate;
diseases or disorders of the pancreas;
Sjogren's syndrome; and,
dry mouth;

34. The method of claim 33, wherein the disease or disorder of the pulmonary system is selected from the group consisting of asthma, chronic obstructive pulmonary disease, lung cancer and pneumonitis.

35. The method of claim 33, wherein the disease or disorder of the gastrointestinal system is selected from the group consisting of gastric ulcers, colitis, inflammatory bowel syndrome, Crohn's disease, gastric and intestinal motility, colon cancer, cancer of the stomach, and cancer of the intestine.

36. The method of claim 33, wherein the disease or disorder of the musculoskeletal system is selected from the group consisting of rheumatoid arthritis, osteoporosis and Paget's disease.

37. The method of claim 33, wherein the disease or disorder of the central nervous system is selected from the group consisting of Alzheimer's disease, encephalitis, meningitis, ischemia and stroke.

38. The method of claim 33, wherein the disease or disorder of the cardiovascular system is selected from the group consisting of hypertension, atherosclerosis, angina, congestive heart failure, myocarditus and cardiac ischemia.

39. The method of claim 33, wherein the disease or disorder of the renal system is selected from the group consisting of glomerular kidney disease, kidney cancer and renal failure.

40. The method of claim 33, wherein the disease or disorder of the hepatic system is selected from the group consisting hepatitis and liver cancer.

41. The method of claim 33, wherein the disease or disorder of the eye is selected from the group consisting of glaucoma, retinitis pigmentosa, cataracts, macular degeneration and dry eye.

42. The method of claim 33, wherein the disease or disorder of the skin is selected from the group consisting of dermatitis, psoriasis, pruritis, dermatitis, eczema, seborrhea, wounds, and melanoma.

43. The method of claim 33, wherein the disease or disorder of the pancreatic system is selected from the group consisting of pancreatitus, pancreatic cancer and diabetes.

44. The method of claim 33, wherein the disease or disorder is dry mouth.

45. The method of claim 33, wherein the disease or disorder is Sjogren's syndrome.

46. The method of claim 33, wherein the disease or disorder is acute or chronic pain.

47. The method of claim 33, wherein the disease or disorder is acute or chronic inflammation.

48. The method of claim 33, wherein the disease of disorder of the prostatic system is selected from the group consisting of benign prostatic hyperplasia and prostatic cancer.

49. The method of claim 33, wherein the disease or disorder of the pancreatic system is selected from the group consisting of pancreatitis, diabetes and pancreatic cancer.

50. A method of screening for a compound that modulates the activity of PAR2 or PAR2 subtype, comprising:
contacting PAR2 or par2-subtype with a compound of claim 1; and,
detecting changes in the activity of the PAR2 or PAR2 subtype.

51. A method of screening for a compound that modulates the activity of PAR2 or PAR2 subtype, comprising:
providing a plurality of cells that express PAR2 or PAR2 subtype;
incubating the cells or a component extracted from the cells with a compound of claim 1; and,
detecting changes in PAR2 activity in the cell.

52. The method of claim 51, wherein the cells over-express the PAR2 or PAR2 subtype.

53. The method of claim 51, wherein the compound is selective for PAR2 or the PAR2 subtype.

54. The compound of claim 1 selected from the group consisting of:
N-[2-hydrazino-2-oxo-1-(4-oxo-3,4-dihydro-1-phthalazinyl)ethyl]benzamide,
N-[2-{(2E)-2-[1-(3-bromophenyl)ethylidene]hydrazino}-2-oxo-1-(4-oxo-3,4-dihydro-1-phthalazinyl)ethyl]benzamide,
N-[2-[(2E)-2-(3-bromobenzylidene)hydrazino]-2-oxo-1-(4-oxo-3,4-dihydro-1-phthalazinyl)ethyl]benzamide,
N-[2-[(2E)-2-(2-furylmethylene)hydrazino]-2-oxo-1-(4-oxo-3,4-dihydro-1-phthalazinyl)ethyl]benzamide,
N-[2-{(2E)-2-[1-(3-methoxyphenyl)ethylidene]hydrazino}-2-oxo-1-(4-oxo-3,4-dihydro-1-phthalazinyl)ethyl]benzamide,
N-[2-[(2E)-2-(2-hydroxybenzylidene)hydrazino]-2-oxo-1-(4-oxo-3,4-dihydro-1-phthalazinyl)ethyl]benzamide,
N-[2-[(2E)-2-(2-bromobenzylidene)hydrazino]-2-oxo-1-(4-oxo-3,4-dihydro-1-phthalazinyl)ethyl]benzamide,
N-[2-{(2E)-2-[1-(4-bromophenyl)ethylidene]hydrazino}-2-oxo-1-(4-oxo-3,4-dihydro-1-phthalazinyl)ethyl]benzamide,
N-[2-{(2E)-2-[1-(2-methoxyphenyl)ethylidene]hydrazino}-2-oxo-1-(4-oxo-3,4-dihydro-1-phthalazinyl)ethyl]benzamide,
N-[2-[(2E)-2-(6-methoxy-2,3-dihydro-1H-inden-1-ylidene)hydrazino]-2-oxo-1-(4-oxo-3,4-dihydro-1-phthalazinyl)ethyl]benzamide,
N-[2-[(2E)-2-(4-fluorobenzylidene)hydrazino]-2-oxo-1-(4-oxo-3,4-dihydro-1-phthalazinyl)ethyl]benzamide
N-[2-{(2E)-2-[1-(2-fluorophenyl)ethylidene]hydrazino}-2-oxo-1-(4-oxo-3,4-dihydro-1-phthalazinyl)ethyl]benzamide,
N-[2-{(2E)-2-[1-(2-bromophenyl)ethylidene]hydrazino}-2-oxo-1-(4-oxo-3,4-dihydro-1-phthalazinyl)ethyl]benzamide,
N-[2-[(2E)-2-(2-fluorobenzylidene)hydrazino]-2-oxo-1-(4-oxo-3,4-dihydro-1-phthalazinyl)ethyl]benzamide,
N-(2-oxo-1-(4-oxo-3,4-dihydro-1-phthalazinyl)-2-{(2E)-2-[3-(trifluoromethyl)-benzylidene]hydrazino}ethyl)benzamide,
N-{2-oxo-1-(4-oxo-3,4-dihydro-1-phthalazinyl)-2-[(2E)-2-(2-pyridinylmethylene)-hydrazino]ethyl}benzamide,
N-{2-oxo-1-(4-oxo-3,4-dihydro-1-phthalazinyl)-2-[(2E)-2-(3-pyridinylmethylene)-hydrazino]ethyl}benzamide,
N-[2-((2E)-2-{[3-chloro-5-(trifluoromethyl)-2-pyridinyl]methylene}-hydrazino)-2-oxo-1-(4-oxo-3,4-dihydro-1-phthalazinyl)ethyl]benzamide,
N-(2-oxo-1-(4-oxo-3,4-dihydro-1-phthalazinyl)-2-{(2E)-2-[1-(3-pyridinyl)-ethylidene]hydrazino}ethyl)benzamide,
N-(2-oxo-1-(4-oxo-3,4-dihydro-1-phthalazinyl)-2-{(2E)-2-[1-(2-pyridinyl)ethylidene]hydrazino}ethyl)benzamide,
N-[2-[(2E)-2-(1,3-dimethylbutylidene)hydrazino]-2-oxo-1-(4-oxo-3,4-dihydro-1-phthalazinyl)ethyl]benzamide,
N-[2-{(2E)-2-[2-(3-methoxyphenyl)-1-methylethylidene]hydrazino}-2-oxo-1-(4-oxo-3,4-dihydro-1-phthalazinyl)ethyl]benzamide,
N-[2-{(2E)-2-[1-(3-bromophenyl)propylidene]hydrazino}-2-oxo-1-(4-oxo-3,4-dihydro-1-phthalazinyl)ethyl]benzamide,
N-[2-{(2E)-2-[1-(3-fluorophenyl)ethylidene]hydrazino}-2-oxo-1-(4-oxo-3,4-dihydro-1-phthalazinyl)ethyl]benzamide,
N-[2-oxo-1-(4-oxo-3,4-dihydro-1-phthalazinyl)-2-((2E)-2-{1-[3-(trifluoromethyl)phenyl]ethylidene}hydrazino)ethyl]benzamide,
N-[2-{(2E)-2-[1-(3-chlorophenyl)ethylidene]hydrazino}-2-oxo-1-(4-oxo-3,4-dihydro-1-phthalazinyl)ethyl]benzamide,
N-[2-{(2E)-2-[1-(3-cyanophenyl)ethylidene]hydrazino}-2-oxo-1-(4-oxo-3,4-dihydro-1-phthalazinyl)ethyl]benzamide,
N-[2-((2E)-2-[1-(3-hydroxyphenyl)ethylidene]hydrazino)-2-oxo-1-(4-oxo-3,4-dihydro-1-phthalazinyl)ethyl]benzamide,
N-[2-{(2E)-2-[1-(3-methylphenyl)ethylidene]hydrazino}-2-oxo-1-(4-oxo-3,4-dihydro-1-phthalazinyl)ethyl]benzamide,
N-[2-{(2E)-2-[1-(4-methyl-2-pyridinyl)ethylidene]hydrazino}-2-oxo-1-(4-oxo-3,4-dihydro-1-phthalazinyl)ethyl]benzamide,
N-[2-((2E)-2-[1-(3-bromophenyl)-2,2-dimethylpropylidene]-hydrazino)-2-oxo-1-(4-oxo-3,4-dihydro-1-phthalazinyl)ethyl]benzamide,
N-[2-{(2E)-2-[1-(3-bromophenyl)-2-methylpropylidene]hydrazino}-2-oxo-1-(4-oxo-3,4-dihydro-1-phthalazinyl)ethyl]benzamide,
N-[2-{(2E)-2-[(3-bromophenyl)(cyclopropyl)methylene]hydrazino}-2-oxo-1-(4-oxo-3,4-dihydro-1-phthalazinyl)ethyl]benzamide,
N-[2-{(2E)-2-[1-(3-bromophenyl)butylidene]hydrazino}-2-oxo-1-(4-oxo-3,4-dihydro-1-phthalazinyl)ethyl]benzamide,
N-[2-[(2E)-2-(2,2-dimethyl-1-phenylpropylidene)hydrazino]-2-oxo-(4-oxo-3,4-dihydro-1-phthalazinyl)ethyl]benzamide,
N-[2-{(2E)-2-[1-(3,5-dimethoxyphenyl)ethylidene]hydrazino}-2-oxo-1-(4-oxo-3,4-dihydro-1-phthalazinyl)ethyl]benzamide,
N-[2-((2E)-2-{1-[3,5-bis(trifluoromethyl)phenyl]ethylidene}-hydrazino)-2-oxo-1-(4-oxo-3,4-dihydro-1-phthalazinyl)ethyl]benzamide,
N-[2-{(2E)-2-[1-(3,5-difluorophenyl)ethylidene]hydrazino}-2-oxo-1-(4-oxo-3,4-dihydro-1-phthalazinyl)ethyl]benzamide,
N-[2-[(2E)-2-(3,5-dibromobenzylidene)hydrazino]-2-oxo-1-(4-oxo-3,4-dihydro-1-phthalazinyl)ethyl]benzamide,
N-[2-{(2E)-2-[1-(3,5-dibromophenyl)ethylidene]hydrazino}-2-oxo-1-(4-oxo-3,4-dihydro-1-phthalazinyl)ethyl]benzamide, and,
N-[2-{(2E)-2-[1-(3,5-dimethylphenyl)ethylidene]hydrazino}-2-oxo-1-(4-oxo-3,4-dihydro-1-phthalazinyl)ethyl]benzamide.

55. A method for treating or preventing a disease or disorder related to PAR2 activity comprising administering a therapeutically effective amount to a patient in need thereof of a compound selected from the group consisting of:
N-[2-hydrazino-2-oxo-1-(4-oxo-3,4-dihydro-1-phthalazinyl)ethyl]benzamide,
N-[2-{(2E)-2-[1-(3-bromophenyl)ethylidene]hydrazino}-2-oxo-1-(4-oxo-3,4-dihydro-1-phthalazinyl)ethyl]benzamide,
N-[2-[(2E)-2-(3-bromobenzylidene)hydrazino]-2-oxo-1-(4-oxo-3,4-dihydro-1-phthalazinyl)ethyl]benzamide,
N-[2-[(2E)-2-(2-furylmethylene)hydrazino]-2-oxo-1-(4-oxo-3,4-dihydro-1-phthalazinyl)ethyl]benzamide,
N-[2-{(2E)-2-[1-(3-methoxyphenyl)ethylidene]hydrazino}-2-oxo-1-(4-oxo-3,4-dihydro-1-phthalazinyl)ethyl]benzamide,
N-[2-[(2E)-2-(2-hydroxybenzylidene)hydrazino]-2-oxo-1-(4-oxo-3,4-dihydro-1-phthalazinyl)ethyl]benzamide,
N-[2-[(2E)-2-(2-bromobenzylidene)hydrazino]-2-oxo-1-(4-oxo-3,4-dihydro-1-phthalazinyl)ethyl]benzamide,
N-[2-{(2E)-2-[1-(4-bromophenyl)ethylidene]hydrazino}-2-oxo-1-(4-oxo-3,4-dihydro-1-phthalazinyl)ethyl]benzamide,
N-[2-{(2E)-2-[1-(2-methoxyphenyl)ethylidene]hydrazino}-2-oxo-1-(4-oxo-3,4-dihydro-1-phthalazinyl)ethyl]benzamide,
N-[2-[(2E)-2-(6-methoxy-2,3-dihydro-1H-inden-1-ylidene)hydrazino]-2-oxo-1-(4-oxo-3,4-dihydro-1-phthalazinyl)ethyl]benzamide,
N-[2-[(2E)-2-(4-fluorobenzylidene)hydrazino]-2-oxo-1-(4-oxo-3,4-dihydro-1-phthalazinyl)ethyl]benzamide,
N-[2-{(2E)-2-[1-(2-fluorophenyl)ethylidene]hydrazino}-2-oxo-1-(4-oxo-3,4-dihydro-1-phthalazinyl)ethyl]benzamide,
N-[2-{(2E)-2-[1-(2-bromophenyl)ethylidene]hydrazino}-2-oxo-1-(4-oxo-3,4-dihydro-1-phthalazinyl)ethyl]benzamide,
N-[2-[(2E)-2-(2-fluorobenzylidene)hydrazino]-2-oxo-1-(4-oxo-3,4-dihydro-1-phthalazinyl)ethyl]benzamide,
N-(2-oxo-1-(4-oxo-3,4-dihydro-1-phthalazinyl)-2-{(2E)-2-[3-(trifluoromethyl)-benzylidene]hydrazino}ethyl)benzamide,
N-{2-oxo-1-(4-oxo-3,4-dihydro-1-phthalazinyl)-2-[(2E)-2-(2-pyridinylmethylene)-hydrazino]ethyl}benzamide,
N-{2-oxo-1-(4-oxo-3,4-dihydro-1-phthalazinyl)-2-[(2E)-2-(3-pyridinylmethylene)-hydrazino]ethyl}benzamide,
N-[2-((2E)-2-{[3-chloro-5-(trifluoromethyl)-2-pyridinyl]methylene}-hydrazino)-2-oxo-1-(4-oxo-3,4-dihydro-1-phthalazinyl)ethyl]benzamide,
N-(2-oxo-1-(4-oxo-3,4-dihydro-1-phthalazinyl)-2-{(2E)-2-[1-(3-pyridinyl)-ethylidene]hydrazino}ethyl)benzamide,
N-(2-oxo-1-(4-oxo-3,4-dihydro-1-phthalazinyl)-2-{(2E)-2-[1-(2-pyridinyl)ethylidene]hydrazino}ethyl)benzamide,
N-[2-[(2E)-2-(1,3-dimethylbutylidene)hydrazino]-2-oxo-1-(4-oxo-3,4-dihydro-1-phthalazinyl)ethyl]benzamide,
N-[2-{(2E)-2-[2-(3-methoxyphenyl)-1-methylethylidene]hydrazino}-2-oxo-1-(4-oxo-3,4-dihydro-1-phthalazinyl)ethyl]benzamide,
N-[2-{(2E)-2-[1-(3-bromophenyl)propylidene]hydrazino}-2-oxo-1-(4-oxo-3,4-dihydro-1-phthalazinyl)ethyl]benzamide,
N-[2-{(2E)-2-[1-(3-fluorophenyl)ethylidene]hydrazino}-2-oxo-1-(4-oxo-3,4-dihydro-1-phthalazinyl)ethyl]benzamide,
N-[2-oxo-1-(4-oxo-3,4-dihydro-1-phthalazinyl)-2-((2E)-2-{(1-[3-(trifluoromethyl)phenyl]ethylidene}hydrazino)ethyl]benzamide,
N-[2-{(2E)-2-[1-(3-chlorophenyl)ethylidene]hydrazino}-2-oxo-1-(4-oxo-3,4-dihydro-1-phthalazinyl)ethyl]benzamide,
N-[2-{(2E)-2-[1-(3-cyanophenyl)ethylidene]hydrazino}-2-oxo-1-(4-oxo-3,4-dihydro-1-phthalazinyl)ethyl]benzamide,
N-[2-{(2E)-2-[1-(3-hydroxyphenyl)ethylidene]hydrazino}-2-oxo-1-(4-oxo-3,4-dihydro-1-phthalazinyl)ethyl]benzamide,
N-[2-{(2E)-2-[1-(3-methylphenyl)ethylidene]hydrazino}-2-oxo-1-(4-oxo-3,4-dihydro-1-phthalazinyl)ethyl]benzamide,
N-[2-{(2E)-2-[1-(4-methyl-2-pyridinyl)ethylidene]hydrazino}-2-oxo-1-(4-oxo-3,4-dihydro-1-phthalazinyl)ethyl]benzamide,
N-[2-{(2E)-2-[1-(3-bromophenyl)-2,2-dimethylpropylidene]-hydrazino}-2-oxo-1-(4-oxo-3,4-dihydro-1-phthalazinyl)ethyl]benzamide,
N-[2-{(2E)-2-[1-(3-bromophenyl)-2-methylpropylidene]hydrazino}-2-oxo-1-(4-oxo-3,4-dihydro-1-phthalazinyl)ethyl]benzamide,
N-[2-{(2E)-2-[(3-bromophenyl)(cyclopropyl)methylene]hydrazino}-2-oxo-1-(4-oxo-3,4-dihydro-1-phthalazinyl)ethyl]benzamide,
N-[2-{(2E)-2-[1-(3-bromophenyl)butylidene]hydrazino}-2-oxo-1-(4-oxo-3,4-dihydro-1-phthalazinyl)ethyl]benzamide,
N-[2-[(2E)-2-(2,2-dimethyl-1-phenylpropylidene)hydrazino]-2-oxo-(4-oxo-3,4-dihydro-1-phthalazinyl)ethyl]benzamide,
N-[2-{(2E)-2-[1-(3,5-dimethoxyphenyl)ethylidene]hydrazino}-2-oxo-1-(4-oxo-3,4-dihydro-1-phthalazinyl)ethyl]benzamide,
N-[2-((2E)-2-{1-[3,5-bis(trifluoromethyl)phenyl]ethylidene}-hydrazino)-2-oxo-1-(4-oxo-3,4-dihydro-1-phthalazinyl)ethyl]benzamide,
N-[2-{(2E)-2-[1-(3,5-difluorophenyl)ethylidene]hydrazino}-2-oxo-1-(4-oxo-3,4-dihydro-1-phthalazinyl)ethyl]benzamide,
N-[2-[(2E)-2-(3,5-dibromobenzylidene)hydrazino]-2-oxo-1-(4-oxo-3,4-dihydro-1-phthalazinyl)ethyl]benzamide,
N-[2-{(2E)-2-[1-(3,5-dibromophenyl)ethylidene]hydrazino}-2-oxo-1-(4-oxo-3,4-dihydro-1-phthalazinyl)ethyl]benzamide; and,
N-[2-{(2E)-2-[1-(3,5-dimethylphenyl)ethylidene]hydrazino}-2-oxo-1-(4-oxo-3,4-dihydro-1-phthalazinyl)ethyl]benzamide.


SEQUENCE LISTING (2 sequences)

SEQ ID NO: 1 - DNA, 1419 bp, Homo sapiens
  PAR2 DNA
ctcgagcggccgccagtgtgatggatatctgcagaattcgccctttgcgtccagtggagctctgagtttcgaatcggtggcggcggattccccgcgcgcccggcgtcggggcttccaggaggatgcggagccccagcgcagcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattgagttttccaggtcctcagatgggaattgcacagtaggatgtggaacctgtttaatgttatgaggacgtgtctgaagggcgaattccagcacactggcggccgtt

SEQ ID NO: 2 - protein, 397 aa, Homo sapiens
  PAR2 Polypeptide
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY